(19) United States
(12) Patent Application Publication
(10) Pub. No.: US 2005/0014827 A1
(43) Pub. Date: Jan. 20, 2005

(54) Synergistic medicament containing flavoring agents and having an antagonistic regenerative and/or protagonist decontamination effect

(76) Inventor: Jorg P. Schur, Wegberg-Dalheim (DE)

Correspondence Address: JENKINS & WILSON, PA, 3100 TOWER BLVD, SUITE 1400, DURHAM, NC, 27707, US

(21) Appl. No.: 10/416,479

(22) PCT Filed: Nov. 9, 2001
(86) PCT No.: PCT/EP01/12974

(30) Foreign Application Priority Data
Nov. 9, 2000 (EP) 00124497.9

(51) Int. Cl. A61K 31/19; A61K 31/045

(52) U.S. Cl. 514/553; 514/557; 514/574; 514/724; 514/730; 514/570

(57) ABSTRACT

The invention relates to medicaments comprising a microbicidal composition consisting of at least two GRAS (Generally Recognized As Safe) aromatic agents or derivatives thereof, and to the use of these compositions for producing decontamination and/or regenerative agents for treating humans and animals.

The present invention relates to medicaments comprising a microbicidal composition of at least two GRAS (generally recognized as safe) flavoring agents or their derivatives, and the use of such compositions for the preparation of decontaminative and/or regenerative agents for the treatment of humans and animals.

### BACKGROUND OF THE INVENTION

The growing number of resistances of microorganisms against known antibiotics requires a continuous further development of the active substances and/or the discovery of new active substances and active principles to ensure sufficient protection for humans and animals. In addition, the use of conventional antibiotics in veterinary medicine (especially in fattening farms) is extremely disputed because the opinion is held that this results in the final member of the food chain, namely humans, to be also excessively subjected to antibiotics or antibiotic metabolites, which may lead to additional resistances.

The increase of defective nutrition, application of anti-infective agents, immunosuppressants, antidepressive, contraceptive, antiallergic agents and the like causes, on the one hand, promotion of resistances in microorganisms and, on the other hand, generation of associated diseases whose symptoms are often not associated causally therewith, because they mimic other clinical pictures. Thus, for example, “fungal infections”, e.g., of the genera Candida and Aspergillus, have significantly increased in recent years, and the above mentioned clinical pictures are with high probability to be considered causally related with Candida and/or Aspergillus contaminations in the mucosa and gastrointestinal tract, all the more so since yeasts and fungi of these genera are also capable of forming mutagenic and cancerogenic toxins, which in turn have effects which in part not only reduce the quality of foods, but are even life-threatening. As early as in 1980, H. J. Preusser (Medical Mycology Zbl. Bact. Suppl. 8) describes the importance of the “pathogenic potential in the genus Candida”, and R. Hurley/De Louvois report on the “ecological aspects of yeast-like fungi of medical importance”. In Wien. Klin. Wochenschr. 91, 826-830 (1979), O. Male/Boltz-Nitulescu already described animal trial studies on the persorption of Candida albicans and the possibility of intestinal triggering of an immune response. Th. Buchner reports in “Pilzinfektionen in der Onkologie”, Schattauer, 1996, on predisposing factors for mycoses: hormonal diseases, therapy: corticosteroids; gastroenterological diseases: therapy immunosuppressants; hematological diseases, therapy: cytostatic agents; immunodeficiencies (including AIDS), therapy: irradiation; malignant tumors, therapy: antibiotics; infectious diseases, chronic diseases, burns, macrosurgery.

Most disinfecting agents and methods of the prior art, such as chemical biocides, disinfectants, antibiotics, chemotherapeutic agents and the like, destroy bacteria, fungi or viruses, for example, by coagulase, cell membrane defects or the like, and mostly in connection with pH value and/or aw value dependence, they have a highly selective effect on microorganisms. Therefore, mechanisms of action of the usual kind lead to resistances (e.g., through the recognition of signals) in microorganisms. When antibody-antigen-like signals of conventional disinfectants are recognized, microorganisms are capable of forming barricade-of-wagon-like defensive strategies with larger attack surfaces which are more difficult to penetrate and thus show a resistance behavior (Bob Shapiro, Monsanto, Calif.).

In selectively acting conventional disinfectants, such as antibiotics, which act as DNA/RNA selective inhibitors by functioning as inhibitors for the formation of new RNA in prokaryotes and eukaryotes, inherited RTFs (resistance transfer factor) r-genes are produced which encode enzymes against certain selective materials for resistance. The animal/human immune system has two recognition mechanisms: a) soluble antibodies (in the form of, for example, proteins, enzymes, amino acids) and cell-bound T-cell receptors, also called “killer cells”, which initiate the lysis of intracellular, pathogenic, infected cells.

The ideal principle of action for the disinfection of microorganisms in the vegetable, animal and human fields is equally effective on fungi, bacteria (Gram-positive/Gram-negative) and viruses and other possible pathogenic cells, e.g., protozoans, without resistance and selection mechanisms in the fields of application, non-toxic, non-mutagenic, non-teratogenic, non-cancerogenic, safe in the workplace and environmentally tolerable, easily prepared and applied. Prerequisites for this are apparently offered by the principle of action based on molecular-physiological transmembrane transport and proton/electron gradient transfer, which causes hydrophilic and lipophilic substances to permeate into the region of integral proteins in microorganism membranes, independently of the pH value under certain preconditions. Thus, lipophilic substances can diffuse through the region of integral proteins. Thus, groups of substances are required which allow for a free equalization of energy levels. They must possess an “energy deficiency” with respect to the “energy excess” of, for example, proteins, RTP in microorganisms, so that an energy equalization takes place with lowering of the energy of, for example, the protein molecules by means of electron transfer with raising the energy of the groups or mixtures of substances which are in “energy deficiency” (attraction=electron transport). Thus, the metabolism of the microorganism can no longer take place and has thus been harmlessly interrupted.

Now, it is the object of the present application to provide a medicament which has bactericidal, fungicidal, virucidal effects, can reduce toxins of, for example, microorganisms, does not tend to resistance formation and offers sufficient protection for humans and animals against pathogenic microorganisms. Surprisingly, it has now been found that specific microbicidal compositions which contain GRAS (generally recognized as safe) flavoring agents as microbicidal components, known as processing aids and additives for foods from WO 96/29895 and WO 98/58540, have suitable microbicidal properties due to which they can also be employed as medicaments for humans and animals.

Due to the fact that the GRAS flavoring agents are, on the one hand, toxicologically safe, i.e., are degraded within or excreted from the body of humans and animals, and on the other hand, these microbicidal compositions exhibit a novel mechanism of action, the problem of resistance formation is clearly reduced, or even excluded, in these medicaments since the mechanism of action has no selective effect and thus all microorganisms are likewise inactivated.

Further, it has been found that flavoring agents or similar groups of substances of natural origin are capable of permeating into the cells of microorganisms without being recognized as “enemies” due to their versatility, permanent variability and capability of a synergistic, symbiotic, protagonistic or antagonistic activity due to versatile active substances which can often be identified only with difficulty in minimum dosages, in contrast to the monosubstances or mixtures according to the invention. Thus, flavoring agents and other similar groups of substances are capable, as synergies, often already as one flavoring agent, to prevent microorganisms from growing (protagonism) or to promote the growth of microorganisms (antagonism).

### SUMMARY OF THE INVENTION

Accordingly, the present invention relates to
(1) a medicament comprising a microbicidal composition which contains at least two GRAS (generally recognized as safe) flavoring agents or their derivatives;
(2) a preferred embodiment of the medicament (1), wherein the microbicidal composition contains:
   (I) one or more GRAS flavor alcohols or their derivatives; and
   (II) one or more flavoring agents selected from
      (II-1) polyphenol compounds (b); and
      (II-2) GRAS flavor acids (c) or their derivatives;
(3) a preferred embodiment of the medicament (1), wherein the microbicidal composition contains the GRAS flavor alcohol benzyl alcohol as a necessary component;
(4) a preferred embodiment of the medicament (1), wherein the microbicidal composition contains at least two GRAS essential oils (i);
(5) a preferred embodiment of the medicament (1), wherein the microbicidal composition contains at least one lipophilic GRAS (generally recognized as safe) flavoring agent and at least one hydrophilic GRAS flavoring agent;
(6) a preferred embodiment of the medicament (1), wherein the microbicidal composition contains at least one GRAS flavoring agent with double bonds (e.g., acids and/or alcohols) or a derivative thereof;
(7) a preferred embodiment of the medicament (1) to (6), which is a decontaminative and/or regenerative agent;
(8) a preferred embodiment of the medicament (7), wherein the medicament is in a form for the following applications: inhalational, oral, intravenous, intramuscular, rectal, contact preparation, internal/external (also mucosa), intraperitoneal, subcutaneous, on/in internal organs (e.g., by endoscopy) in the form of tablets, liquid, gas, powder, injection, infusion, suppository, spray, ointments or plasters;
(9) use of the microbicidal compositions as defined in (1) to (6) for the preparation of a decontaminative and/or regenerative agent, especially for the preparation of an antibiotic, cytostatic agent or agent for the treatment of: overweight (adenovirucide), rheumatism, dermatoses, gastritis, gastro-intestinal diseases, bronchial diseases, depressions, arthritis, mucosal diseases, impotence, defective concentration, psychic disorders, migraine, sleep disturbance (i.e., vegetative symptoms), gastrointestinal symptoms, allergies and skin diseases, joint diseases, genital and hormonal disorders; infections, cancer diseases and immune insufficiency;
(10) use of a GRAS flavoring agent or a derivative thereof for the preparation of a regenerative agent;
(11) a food supplement or animal feed comprising a GRAS flavoring agent or a derivative thereof and/or a microbicidal composition as defined in (1) to (6); and
(12) a method for the treatment of the diseases/symptoms as defined under (9) in humans and animals, comprising the administration of a GRAS flavoring agent or derivative thereof and/or a microbicidal composition as defined in (1) to (6) to humans and animals in need of such a treatment.

The microbicidal compositions as defined above under (1) to (6) exhibit (A) GRAS flavoring agent synergies (protagonisms) and may therefore be employed in decontaminative medicaments
(a) for the disinfection of bacteria, bacilli, viruses, yeasts and fungi (general antibiotic activity—decontaminative agent; GRAS flavoring agent synergies, i.e., protagonisms); or
(b) for the disinfection of, e.g., mycoses (yeasts, fungal infections and general decontaminations); on the one hand as (b) antimycotic and decontaminative agent or, on the other hand, as
(c) multi-step application for eliminating the causes of the diseases developing therefrom (e.g., mycoses or other infections/contaminations which may lead to them) (general antimycotic activity and/or multi-step application; step 1—decontaminative agent).

In addition, the microbicidal compositions as defined above under (1) to (6) exhibit:
(B) GRAS flavoring agent synergies (antagonisms); therefore, the medicaments may also be employed as regenerative agents for maintaining or supporting or promoting the growth of microorganisms necessary to life (e.g., in the gastrointestinal tract, small intestine, oral mucosa, genital mucosa), both
(d) as an agent having a general regenerative effect; and/or
(e) in multi-step applications (e.g., as step 2 after decontaminative application in step 1).
(C) GRAS flavoring agent synergies of the compositions as defined under (1) to (6) are capable of
(f) reducing toxins from microorganisms (with no formation of resistance, as in applications (A) and (B)).
(D) additional importance is gained by the GRAS flavoring agent synergy (antagonism) when
(g) “benign” microorganisms necessary to life (individual mixtures or synergies, symbiotic agent) (live, preserved, killed, cells or the like) are supplied to the human or animal organisms in a regenerative way to favor their positive growth and to promote the access to body cells, their permeation or association, or even to enable it.

The use of these agents (A) and (C), i.e., decontaminative agents, and (B) and (D), i.e., regenerative agents, is effected in/on the human or animal body and may be applied individually, in combinations or in a multi-step method. It is solid, liquid or gaseous. When applied orally, it may be supplemented with inert additives/carrier agents/fillers/supplements to display its selective activity and the like in the corresponding passages of the stomach, small intestine and large intestine or ileum, also with timers combined therewith, i.e., functionally inert substances for the well-aimed application fixed in time. Other applications include: i.m., i.v., inhalational, oral, intravenous, intramuscular, rectal, contact preparation, internal/external (also mucosa), intraperitoneal, subcutaneous, on/in internal organs (e.g., by endoscopy) in the form of tablets, liquid, gas, powder, injection, infusion, suppository, spray, ointments, plasters.

### DETAILED DESCRIPTION OF THE INVENTION

In the following, the microbicidal compositions (1) to (6) according to the invention are further described in more detail:

The mentioned GRAS flavoring agents are recognized by the FDA authority as commercially safe for use in foods (GRAS=generally recognized as safe in food). The mentioned GRAS flavoring agents are the compounds mentioned in the FEMA/FDA GRAS Flavour Substances Lists GRAS 3-15 Nos. 2001-3905 (as of 2000). This list contains natural and synthetic flavoring agents approved by the American public health authority, FDA, for use in foodstuffs: FDA Regulation 21 CFR 172.515 for synthetic flavoring agents (Synthetic Flavoring Substances and Adjuvants) and FDA Regulation 21 CFR 182.20 for natural flavoring agents (Natural Flavoring Substances and Adjuvants).

The GRAS flavoring agents of the microbicidal composition of the medicaments (1) to (6) of the present invention are preferably selected from (a) GRAS flavor alcohols or their derivatives, (b) GRAS polyphenols, (c) GRAS acids or their derivatives, (d) GRAS phenols or their derivatives, (e) GRAS esters, (f) GRAS terpenes, (g) GRAS acetals, (h) GRAS aldehydes and (i) GRAS essential oils.

In detail, the following GRAS flavor alcohols (a) may be employed, for example: benzyl alcohol, acetoin (acetylmethylcarbinol), ethyl alcohol (ethanol), propyl alcohol (1-propanol), iso-propyl alcohol (2-propanol, isopropanol), propylene glycol, glycerol, n-butyl alcohol (n-propyl carbinol), iso-butyl alcohol (2-methyl-1-propanol), hexyl alcohol (hexanol), L-menthol, octyl alcohol (n-octanol), cinnamyl alcohol (3-phenyl-2-propene-1-ol), α-methylbenzyl alcohol (1-phenyl-ethanol), heptyl alcohol (heptanol), n-amyl alcohol (1-pentanol), iso-amyl alcohol (3-methyl-1-butanol), anisalcohol (4-methoxybenzyl alcohol, p-anisalcohol), citronellol, n-decyl alcohol (n-decanol), geraniol, β-γ-hexanol (3-hexenol), lauryl alcohol (dodecanol), linalool, nerolidol, nonadienol (2,6-nonadiene-1-ol), nonyl alcohol (nonanol-1), rhodinol, terpineol, borneol, clineol (eucalyptol), anisole, cuminyl alcohol (cuminol), 10-undecen-1-ol, 1-hexadecanol. As said derivatives, both natural and synthetic (naturally occurring or not) derivatives can be employed. Suitable derivatives include, for example, the esters, ethers and carbonates of the above mentioned GRAS flavor alcohols. Particularly preferred GRAS flavor alcohols are benzyl alcohol, 1-propanol, glycerol, propylene glycol, n-butyl alcohol, citronellol, hexanol, linalool, acetoin and their derivatives.

As polyphenols (b), the following polyphenols may be employed, in particular: catechol, resorcinol, hydroquinone, phloroglucinol, pyrogallol, cyclohexane, usnic acid, acylpolyphenols, lignins, anthocyans, flavones, catechols, gallic acid derivatives (e.g., tannins, gallotannin, tannic acids, gallotannic acids), carnosol, carnosolic acid (including their derivatives, such as (2,5-dihydroxyphenyl)carboxylic and (2,5-dihydroxyphenyl)alkylenecarboxylic substitutions, salts, esters, amides), caffeic acid and its esters and amides, flavonoids (e.g., flavone, flavonol, isoflavone, gossypetin, myricetin, robinetin, apigenin, morin, taxifolin, eriodictyol, naringin, rutin, hesperidin, troxerutin, chrysin, tangeritin, luteolin, catechols, quercetin, fisetin, kaempferol, galangin, rotenoids, aurones, flavonols, diols), extracts, e.g., from Camellia, Primula. Further, their possible derivatives, e.g., salts, acids, esters, oxides and ethers, may also be used. A particularly preferred polyphenol is tannin (a GRAS compound).

As GRAS acids (c), the following acids may be used, for example: acetic acid, aconitic acid, adipic acid, formic acid, malic acid (1-hydroxysuccinic acid), capronic acid, hydrocinnamic acid (3-phenyl-1-propionic acid), pelargonic acid (nonanoic acid), lactic acid (2-hydroxypropionic acid), phenoxyacetic acid (glycolic acid phenyl ether), phenylacetic acid (α-toluenic acid), valeric acid (pentanoic acid), iso-valeric acid (3-methylbutyric acid), cinnamic acid (3-phenylpropenoic acid), citric acid, mandelic acid (hydroxyphenylacetic acid), tartaric acid (2,3-dihydroxybutanedioic acid; 2,3-dihydroxysuccinic acid), fumaric acid, tannic acid and their derivatives.

Suitable derivatives according to the present invention are esters (e.g., C₁₋₆-alkyl esters and benzyl esters), amides (including N-substituted amides) and salts (alkali, alkaline earth and ammonium salts) of the above mentioned acids. According to the present invention, the term “derivatives” also encompasses modifications of the side-chain hydroxy functions (e.g., acyl and alkyl derivatives) and modifications of the double bonds (e.g., the perhydrogenated and hydroxylated derivatives of the mentioned acids).

As GRAS phenols (d), the following phenol compounds may be employed: thymol, methyleugenol, acetyleugenol, safrol, eugenol, isoeugenol, anethole, phenol, methylchavicol (estragol; 3-(4-methoxyphenyl)-1-propene), carvacrol, α-bisabolol, fornesol, anisole (methoxybenzene), propenylguaethol (5-propenyl-2-ethoxyphenol) and their derivatives. Derivatives according to the present invention are compounds in which the phenolic hydroxy group has been esterified or etherified.

As GRAS esters (e), allicin and the following acetates may be used, for example: iso-amyl acetate (3-methyl-1-butyl acetate), benzyl acetate, benzylphenyl acetate, n-butyl acetate, cinnamyl acetate (3-phenylpropenyl acetate), citronellyl acetate, ethyl acetate (acetic ester), eugenol acetate (acetyleugenol), geranyl acetate, hexyl acetate (hexanyl ethanoate), hydrocinnamyl acetate (3-phenylpropyl acetate), linalyl acetate, octyl acetate, phenylethyl acetate, terpinyl acetate, triacetin (glyceryl triacetate), potassium acetate, sodium acetate and calcium acetate. Further suitable esters are the ester derivatives of the above defined acids (c).

As terpenes (f), there may be used, in particular, camphor, limonene and β-caryophyllene.

The acetals (g) which can be used include, in particular, acetal, acetaldehyde dibutyl acetal, acetaldehyde dipropyl acetal, acetaldehyde phenethyl propyl acetal, cinnamic aldehyde ethylene glycol acetal, decanal dimethyl acetal, heptanal dimethyl acetal, heptanal glyceryl acetal and benzaldehyde propylene glycol acetal.

As aldehydes (h), there may be used, in particular, acetaldehyde, anisaldehyde, benzaldehyde, iso-butyl aldehyde (methyl-1-propanal), citral, citronellal, n-caprylic aldehyde (n-decanal), ethylvanillin, furfural, heliotropin (piperonal), heptyl aldehyde (heptanal), hexyl aldehyde (hexanal), 2-hexenal (β-propylacrolein), hydrocinnamic aldehyde (3-phenyl-1-propanal), lauryl aldehyde (dodecanal), nonyl aldehyde (n-nonanal), octyl aldehyde (n-octanal), phenylacetaldehyde (1-oxo-2-phenylethane), propionaldehyde (propanal), vanillin, cinnamic aldehyde (3-phenylpropenal), perillaldehyde and cuminaldehyde.

As GRAS essential oils (i), the following essential oils and/or alcoholic or glycolic extracts or extracts obtained by CO₂ high-pressure processes from the mentioned plants may be employed, in particular:
(i1) oils or extracts having a high content of alcohols: melissa, coriander, cardamon, eucalyptus;
(i2) oils or extracts having a high content of aldehydes: Eucalyptus citriodora, cinnamon, lemon, lemon grass, melissa, citronella, lime, orange;
(i3) oils or extracts having a high content of phenols: origanum, thyme, rosemary, orange, clove, fennel, camphor, mandarin, anise, cascarilla, estragon and pimento;
(i4) oils or extracts having a high content of acetates: lavender;
(i5) oils or extracts having a high content of esters: mustard, onion, garlic;
(i6) oils or extracts having a high content of terpenes: pepper, bitter orange, caraway, dill, lemon, peppermint, nutmeg;
(i7) oils or extracts having a high content of acids: olibanum.

In the preferred embodiment (2) of the present invention, component (I) contains one or more GRAS flavor alcohols or their derivatives. According to the invention, it is preferred to use one, two or three GRAS flavor alcohol.

The mixing ratio of component (I) to components (II) is preferably between 10,000:1 and 1:10,000, more preferably between 1000:1 and 1:1000, and even more preferably between 100:1 and 1:100.

In a preferred embodiment, the microbicidal composition of the medicament (2) contains:
(I1) benzyl alcohol as a necessary component; and optionally
(I2) one or more further GRAS flavor alcohols (a) or their derivatives; and
(II-1) one or more polyphenol compounds (b); and/or
(II-2) one or more GRAS acids (c) or their derivatives.

Suitable amounts of components (I-1), (I-2), (II-1) and (II-2) are:
from 0.1 to 99% by weight, preferably from 0.1 to 75% by weight, of benzyl alcohol;
from 0 to 99.8% by weight, preferably from 0.01 to 99% by weight, of component (I-2);
from 0 to 25% by weight, preferably from 0.01 to 10% by weight, of component (II-1); and/or
from 0 to 70% by weight, preferably from 0.01 to 30% by weight, of component (II-2).

The microbicidal composition of the medicament (2) may further contain the above defined GRAS flavoring agents (d) to (i), wherein their proportion in the microbicidal composition is preferably smaller than or equal to 25% by weight, preferably within a range of from 0.001 to 9% by weight. Preferred among the further GRAS flavoring agents are the phenols (d), aldehydes (h) and essential oils (i).

Particularly preferred according to the present invention are those microbicidal compositions in which the microbicidally active component exclusively consists of GRAS flavoring agents, i.e., does not contain any “derivatives” of the GRAS flavoring agents. As an example of such a composition, there may be mentioned a mixture of benzyl alcohol, one or two of the above mentioned GRAS flavor alcohols (a) and tannin. Such a mixture preferably contains from 80 to 98% by weight of benzyl alcohol and from 1 to 10% by weight of tannin. Another example of a preferred composition is a mixture of two alcohols (a), a polyphenol (especially tannin) and an essential oil (i), especially the phenolic essential oil (i3).

In embodiment (4) of the present invention, the microbicidal composition contains at least two GRAS essential oils (i). These are preferably the above mentioned essential oils and/or alcoholic or glycolic extracts or extracts obtained by CO₂ high-pressure processes, (i1) to (i6). In addition, the microbicidal composition may contain further GRAS flavoring agents, such as alcohols (a), polyphenol compounds (b), acids (c), phenols (d), esters (e), terpenes (f), acetals (9), aldehydes (h), their derivatives and/or flavor carrier agents (j).

The GRAS flavoring agents (a) to (h) and their derivatives are the above defined GRAS compounds. However, for distinguishing from the microbicidal composition of embodiment (2), it is to be considered that the microbicidal composition of medicament (4), when it contains a GRAS flavor alcohol (a), preferably does not contain any polyphenol compounds (b) and/or GRAS flavor acids (c).

As said flavor carrier agents (j), there may be used both some of the above listed GRAS flavoring agents having flavor carrier properties and suitable non-GRAS compounds. Preferred flavor carrier agents include lecithins, 1,2-propylene glycol (x), glycerol (x), glycerol acetates, ethyl citrates, ethyl lactate, benzyl alcohol (x), mono- and diglycerides of edible fatty acids, also esterified with acetic acid, lactic acid, citric acid, tartaric acid, alginic acid (x), sodium alginate, potassium alginate, calcium alginate (x), agar-agar, carrageen, locust bean gum, guar gum, tragacanth, gum arabic, xanthan, pectins, methylcellulose, carboxymethylcellulose, acetylated distarch phosphate, starch acetate esterified with acetic anhydride, acetylated distarch adipate, calcium and magnesium stearates, sodium, potassium and calcium acetates, sodium, potassium and calcium lactates (x), sodium, potassium and calcium citrates, sodium, potassium, calcium and magnesium carbonates, sorbitol, colloidal silica (x), dicalcium orthophosphate, foods, food additives, food supplements, food raw materials, fodders, fodder additives, fodder supplements, fodder raw materials; of these, propylene glycol, benzyl alcohol, glycerol, alginates, lactates, silica and alginic acid are particularly preferred. The microbicidal composition of embodiment (4) preferably contains:
from 0.01 to 20% by weight, preferably from 0.1 to 10% by weight, of GRAS essential oils (i);
from 0 to 80% by weight, preferably from 0.01 to 40% by weight, of GRAS flavoring agents (a) to (h); and
from 0 to 80% by weight, preferably from 0.01 to 50% by weight, of flavor carrier agents (j).

Particularly preferred microbicidal compositions according to embodiment (4) of the present invention are those which contain at least three GRAS essential oils (c) and/or those in which the further GRAS flavoring agents are anisole and quercitin. The latter compositions are particularly preferred. This particularly preferred microbicidal composition contains:
from 0.1 to 100% by weight, preferably from 0.5 to 80% by weight, of GRAS essential oils (i);
from 0 to 20% by weight, preferably from 0.01 to 10% by weight, of anisole; and
from 0 to 20% by weight, preferably from 0.01 to 10% by weight, of quercitin.

According to embodiment (5) of the present invention, the microbicidal composition contains at least one lipophilic and at least one hydrophilic GRAS flavoring agent (however, it is to be noted that hydrophilic-hydrophilic and lipophilic-lipophilic GRAS flavoring agent combinations also have excellent microbicidal activities). The hydrophilic GRAS flavoring agent may be a hydrophilic alcoholic GRAS flavoring agent (ah) and/or a hydrophilic non-alcoholic GRAS flavoring agent. The proportion of hydrophilic alcoholic GRAS flavoring agents may be up to 99% by weight of the composition and is preferably from 30 to 98% by weight, more preferably from 80 to 95% by weight. The proportion of hydrophilic non-alcoholic GRAS flavoring agents in the composition may be up to 90% by weight and is preferably from 0.1 to 50% by weight. Preferred are those compositions which further contain benzyl alcohol and/or a polyphenol compound (b) in addition to the mentioned hydrophilic compounds.

The hydrophilic alcoholic GRAS flavoring agents (ah) are monovalent or polyvalent alcohols having from 2 to 10 carbon atoms, preferably from 2 to 7 carbon atoms. Particularly preferred compounds are 1-propanol, glycerol, propylene glycol and acetoin. Hydrophilic non-alcoholic GRAS flavoring agents are selected from organic acids (cₕ) having from 1 to 15 carbon atoms and physiologically acceptable salts thereof, hydrophilic acetates (eₕ) and hydrophilic aldehydes (hₕ). Preferred organic acids (cₕ) are those having from 2 to 10 carbon atoms, especially acetic acid, aconitic acid, formic acid, malic acid, lactic acid, phenylacetic acid, citric acid, mandelic acid, tartaric acid, fumaric acid, tannic acid, hydrocinnamic acid and their physiologically acceptable salts. The hydrophilic acetate (cₕ) is preferably selected from allicin, triacetin, potassium acetate, sodium acetate and calcium acetate, and the hydrophilic aldehyde (hₕ) is preferably selected from furfurol, propionaldehyde and vanillin.

In the composition employed in embodiment (5) of the medicament according to the invention, the lipophilic GRAS flavoring agents are preferably selected from (aₗ) lipophilic GRAS flavor alcohols or their derivatives, (b) polyphenol compounds, (cₗ) lipophilic GRAS flavor acids or their derivatives, (d) phenols or their derivatives, (eₗ) lipophilic esters, (f) terpenes, (g) acetals, (hₗ) lipophilic aldehydes and (i) essential oils. The microbicidal composition preferably contains two of the mentioned lipophilic GRAS flavoring agents.

Suitable lipophilic GRAS flavor alcohols (aₗ) among the above defined alcohols (a) are, in particular:
aromatic GRAS flavor alcohols, comprising benzyl alcohol, 2-phenylethanol, 1-phenylethanol, cinnamyl alcohol, hydrocinnamyl alcohol, 1-phenyl-1-propanol and anisalcohol; and aliphatic GRAS flavor alcohols, comprising n-butyl alcohol, iso-butyl alcohol, hexyl alcohol, L-menthol, octyl alcohol, heptyl alcohol, n-amyl alcohol, iso-amyl alcohol, anisalcohol, citronellol, n-decyl alcohol, geraniol, β-γ-hexanol, lauryl alcohol, linalool, nerolidol, nonadienol, nonyl alcohol, rhodinol, terpineol, borneol, clineol, anisole, cuminyl alcohol, 10-undecene-1-ol and 1-hexadecanol and their derivatives. The aromatic GRAS flavor alcohols and especially benzyl alcohol are preferred.

The lipophilic polyphenol compound (b), phenols or their derivatives (d), terpenes (f), acetals (g) and essential oils (i) in the composition of medicament (8) are preferably the above defined compounds (b), (d), (f), (g) and (i). The lipophilic GRAS flavor alcohols or their derivatives (cₗ), lipophilic esters (eₗ) and lipophilic aldehydes comprise all the specifically mentioned acids, esters and aldehydes except for the compounds (cₕ), (eₕ) and (hₕ) specifically mentioned above.

In a preferred embodiment of medicament (5), the microbicidal composition contains either
(i) two lipophilic GRAS flavor alcohols (aₗ), but no benzyl alcohol and no polyphenol compounds (b); or
(ii) benzyl alcohol and/or a polyphenol compound (b), but no further GRAS flavor alcohols.

It is particularly preferred for the microbicidal composition to contain exclusively non-alcoholic hydrophilic GRAS flavoring agents, especially exclusively a hydrophilic GRAS flavor acid (cₕ), and for the microbicidal/antiparasitic composition to contain from 0.01 to 99% by weight, preferably from 0.1 to 90% by weight, of benzyl alcohol or polyphenol compounds (b) and from 0.01 to 50% by weight, preferably from 0.1 to 30% by weight, of hydrophilic non-alcoholic GRAS flavor agents.

In a further preferred embodiment of medicament (5), the microbicidal composition contains
(III) one or more GRAS flavor alcohols (aₗ) or their derivatives; and
(IV) one or more flavoring agents selected from
   polyphenol compounds (b) (component IV-1) and
   lipophilic GRAS flavor acids or their derivatives (cₗ) (component IV-2).

Preferably, this composition contains from 0.1 to 99% by weight, preferably from 0.5 to 99% by weight, of component (III), from 0 to 25% by weight, preferably from 0.01 to 10% by weight, of component (IV-1), and from 0 to 70% by weight, preferably from 0.01 to 30% by weight, of component (IV-2).

In addition, the microbicidal composition may contain further GRAS flavoring agents selected from (d) phenols or their derivatives, (eₗ) lipophilic esters, (f) terpenes, (g) acetals, (hₗ) lipophilic aldehydes and (i) essential oils.

Further, it is preferred for component (III) of the microbicidal composition to contain benzyl alcohol as a necessary component and optionally one or more further lipophilic GRAS flavor alcohols or their derivatives (aₗ). Preferably, this microbicidal composition contains:
from 0.1 to 99% by weight, preferably from 0.1 to 75% by weight, of benzyl alcohol;
from 0 to 99.8% by weight, preferably from 0.01 to 99% by weight, of component (aₗ); and
from 0 to 25% by weight, preferably from 0.01 to 10% by weight, of component (IV-1);
from 0 to 70% by weight, preferably from 0.01 to 30% by weight, of component (IV-2).

This microbicidal composition may also contain further lipophilic GRAS flavoring agents (d) to (i) as defined above, preferably from 0.001 to 25% by weight, more preferably from 0.01 to 9% by weight, of the further flavoring agents (d) to (i). Said further lipophilic GRAS flavoring agents are more preferably phenols (d) and/or essential oils (i).

In a further particularly preferred embodiment of medicament (5), component (III) of the microbicidal composition consists of two lipophilic GRAS flavor alcohols, and component (IV) contains at least one polyphenol compound (b). Said polyphenol compound (b) is preferably tannin, particularly preferred being a composition which contains from 20 to 98% by weight of benzyl alcohol and from 0.01 to 10% by weight of tannin.

Particularly preferred according to the present invention are microbicidal compositions whose microbicidally active component exclusively consists of GRAS flavoring agents, i.e., does not contain any “derivatives” of the GRAS flavoring agents. As an example of such a composition of embodiment (5) of the invention, there may be mentioned a mixture of benzyl alcohol, one or two of the above mentioned GRAS flavor alcohols (aₗ) and tannin. Such a mixture preferably contains from 0.1 to 98% by weight of benzyl alcohol and from 0.01 to 10% by weight, preferably from 1 to 10% by weight, of tannin. Another example of a preferred composition is a mixture of two alcohols, a polyphenol (especially tannin) and an essential oil (especially a phenolic essential oil, component (i3)).

In embodiment (6) of the present invention, the microbicidal composition contains at least one GRAS flavoring agent having a double bond (Δ) or a derivative thereof, preferably at least two such compounds. Examples of compound (Δ) include unsaturated GRAS alcohols (a_{Δ}), such as cinnamyl alcohol, citronellol, 3-hexenol, nonadienol and 10-undecen-1-ol, unsaturated GRAS acids (c_{Δ}), such as cinnamic acid and fumaric acid, unsaturated GRAS esters (d_{Δ}), such as cinnamic acid esters (e.g., ethyl cinnamate and propyl cinnamate), cinnamyl acetate and citronellyl acetate, unsaturated GRAS acetals (g_{Δ}), such as cinnamic aldehyde ethylene glycol acetal, and unsaturated GRAS aldehydes (h_{Δ}), such as cinnamic aldehyde and citronellal. The proportion of compounds (Δ) in composition (6) is preferably within a range of from 0.01 to 70% by weight, more preferably within a range of from 0.1 to 30% by weight. As further GRAS flavoring agents, these compositions preferably contain essential oils (i) and/or the above defined hydrophilic GRAS flavoring agents.

The following are preferred compositions of embodiments (1) to (6). The stated amounts, which are in percent by weight unless otherwise specified, are only particularly preferred embodiments of the respective compositions. Particularly preferred embodiments are those designated with “BHQ”; the effectiveness of these special compositions is shown in the Examples.

1. Compositions comprising at least two GRAS flavoring agents or their derivatives:
45% anisalcohol (a), 35% borneol (a), 20% rhodinol (a) (BHQ-1);
82% valeric acid (c), 18% camphor (i3);
10% malic acid (c), 90% acetoin (a);
12% α-bisabolol (d), 18% geranyl acetate (a), 20% rhodinol (a).

2. Compositions comprising one or more GRAS flavor alcohols (a) and one or more GRAS flavors:
65% propylene glycol (a), 10% caffeic acid (b), 10% tannin (b), 5% resveratrol (b), 10% lactic acid (c) (BHQ-A);
70% propylene glycol (a), 20% anisalcohol (a), 5% quercetin (b), 5% tannin (b) (BHQ-C);
82% L-menthol (a), 8% anisol (d), 7% citronellol (a), 3% safrol (d) (BHQ-2);
98% propylene glycol (a), 2% allicin (e) (BHQ-3).

3. Compositions comprising benzyl alcohol as a necessary component:
79% benzyl alcohol (a), 20% geraniol (a), 1% tannin (b) (BHQ-AFC-1);
95% benzyl alcohol (a), 5% mandelic acid (c) (BHQ-AFC-2);
70% benzyl alcohol (a), 30% catechol (b) (BHQ-AFC-3);
1% benzyl alcohol (a), 88% propylene glycol (a), 1% tannin (b), 10% lactic acid (c) (BHQ-B).

4. Compositions comprising at least two GRAS essential oils (i):
90% bitter orange (i6), 10% cinnamon (i2);
6% origanum (i3), 8% coriander (i1), 7% citric acid (c), 79% propylene glycol (j) (BHQ-6);
1% lavender (i4), 1% anise (i3), 2% safrol (d), 96% xanthan (j);
5% cardamon (il), 5% anise (i3), 10% eucalyptus (i2), 80% carrier agents (j), especially alginic acid.

5. Compositions comprising at least one lipophilic and at least one hydrophilic GRAS flavoring agent:
25% cinnamyl alcohol (aₗ), 25% linalool (aₗ), 50% glycerol (aₕ) (BHQ-5);
25% cinnamyl alcohol (aₗ), 25% linalool (aₗ), 50% lactic acid (cₕ);
50% benzyl alcohol (aₗ), 50% glycerol (aₕ);
50% benzyl alcohol (aₗ), 50% lactic acid (cₕ);
50% tannin (b), 50% glycerol (aₕ);
50% tannin (b), 50% lactic acid (cₕ);
45% cinnamyl alcohol (aₗ), 40% linalool (aₗ), 10% tannin (b), 5% vanillin (hₕ).

6. Compositions comprising at least one GRAS flavoring agent having a double bond:
70% cinnamic aldehyde (h), 30% origanum (i3);
20% eugenol (d), 50% citronellol (a), 20% citronellal (h), 10% ethylcinnamate (e) (BHQ-V);
10% cinnamic aldehyde (h), 90% glycerol (a) (BHQ-4).

The particularly preferred compositions BHQ-1 to BHQ-6 can be employed, in particular, as decontaminative agents; BHQ-A, -B and -C can be employed, in particular, as regenerative agents; BHQ-AFC+MT can be employed, in particular, as an anti-mycotoxin; BHQ-AFC can be employed, in particular, as a fungicide; and BHQ-V can be employed, in particular, as a virucide. However, it is to be considered that:
(i) all the mentioned BHQs can be employed as both decontaminative and regenerative agents;
(ii) low to lowest dosages of BHQ decontaminative agents may turn them into regenerative agents;
(iii) the above described properties of the BHQs are predominantly the properties observed in the tests performed (suspension and/or inhibition halo tests) and are dependent on the dosages which have been selected therein or, in part, which are prescribed.

In addition to components (a) to (i) and flavor carrier agents (j), the medicaments (1) to (6) according to the invention may contain further commercially available and pharmacologically acceptable compounds and carrier materials (k), such as alcohols (k1), emulsifiers (k2), stabilizers (k3), antioxidants (k4), preservatives (k5), solvents (k6), ointments (k7), carriers (k8) including those having a “timer” function (i.e., disintegration=effect at the designated site) etc. The proportion of components (k) in the microbicidal composition depends on the dosage form of the medicament and may be up to 95% by weight, is preferably smaller than 10% by weight and is preferably within a range of from 0.1 to 5% by weight. Thus, the amount of additives is very low in inhalation agents (aerosols) with a content of microbicidal composition of as much as over 90% by weight of the aerosol, but is clearly larger, for example, when the medicament is applied orally, intravenously or intramuscularly in which case the content of microbicidal composition is usually within a range of from 0.1 to 20% by weight, but in some applications, it may also be up to 95 and even 100% by weight of the functional composition. Similarly, the mutual proportion of compounds (k) depends on the dosage form of the medicament.

According to the invention, the alcohols (k1) are monovalent or polyvalent alcohols having from 2 to 10 carbon atoms, preferably from 2 to 7 carbon atoms, not including the GRAS alcohols (a). Preferably, GRAS flavor alcohols (a) and other alcohols (k1) are employed in such amounts that their mixing ratio is between 1000:1 and 1:1000, especially between 100:1 and 1:100, more preferably between 10:1 and 1:10.

The medicament may be in a solid, liquid or gaseous form to be administered to humans and animals. The medicament may be an inhalational, oral, intravenous, intramuscular, rectal agent, contact preparation, internal/external (also mucosa), intraperitoneal, subcutaneous agent, displaying its activity on/in internal organs (e.g., by endoscopy) in the form of tablets, liquid, gas, powder, injection, infusion, suppository, spray, ointments, plasters. The medicament may be for preventive administration and for the treatment of acute affliction.

The medicament of the present invention may be employed, for example, as an inhalational agent, especially for inhaling in respiratory diseases, especially in treatments of pathogens of pneumonia or in the mucosal region against mycoses and similar contaminations.

On the other hand, such inhalational agents can also be employed for prevention in stables of factory farming (e.g., chickens, pigs, cattle) to counteract bronchial diseases, which means a reduced uptake of fodder and thus loss of weight. The possible devices for nebulizing stables with such an inhalational agent are described in the application DE 199 31 185.4. The nebulizing is effected in such a way that the concentration of the microbicidal composition is from 0.01 to 1 ml per m³ of air, especially from 0.01 to 0.1 ml per m³ of air. In exchanging air systems in which an hourly recirculation occurs, the dosage is to be adjusted to result in from 0.01 to 1 ml/m³/h, especially from 0.02 to 0.1 ml/m³/h, of microbicidal composition (1) to (6).

From a toxicological point of view, it is particularly preferred that the microbicidal compositions of the medicaments according to the invention exclusively consist of GRAS flavoring agents. Further, especially in the use of the medicament according to the invention for nebulizing the stables in factory farming, care should be taken that the microbicidal composition be free from ethanol and isopropanol or free from harmful doses of ethanol and isopropanol, since the uptake (inhalation of major amounts) of these substances is harmful to health. In addition, when such compounds are used, there may be danger of explosion.

The decontaminative activity of the medicaments according to the invention is based on the following new principle of action: The composition permits penetration of the components into the microorganism and thereby prevents its proliferation, but does not destroy it. The regenerative activity permits penetration into the microorganism and/or the body cell to thereby stabilize and/or proliferate and/or permeate “benign” microorganisms.

In addition, the medicament of the present invention may also be employed as a cytostatic agent, antiallergic agent, agent for the treatment of: overweight, rheumatism, dermatoses, gastritis, gastrointestinal diseases, bronchial diseases, diseases of the genital and urinary tracts, depressions, arthritis, mucosal diseases, impotence, defective concentration, psychic disorders, lack of drive, diseases of the internal organs, menstrual disorders, migraine, sleep disturbance, i.e., vegetative symptoms, gastrointestinal symptoms, allergies and skin diseases, joint diseases, genital and hormonal disorders, infections, cancers and immune insufficiency.

According to embodiment (9), the invention further relates to the use of the above defined microbicidal compositions (1) to (6) for the preparation of medicaments, e.g., an inhalational agent for the treatment of respiratory diseases or mucosal contaminations in humans and animals, and for the preparation of antibiotics for humans and animals. The term “antibiotics” as used in the present invention means medicaments having a microbicidal decontaminative activity.

In the food supplements and animal feeds (11) according to the invention, the proportion of microbicidal composition (1) to (6) is preferably from 0.1 to 20% by weight, but in some applications, it may also be up to 95 or even 100% by weight of the functional composition.

Finally, the invention relates to methods for the treatment of humans and animals (12), e.g., the treatment of respiratory diseases in humans and animals, comprising the inhalational administration of the above defined microbicidal compositions; methods for the systemic treatment of humans and animals comprising the administration of the microbicidal compositions by inhalation, orally, intravenously, intramuscularly, rectally, as a contact preparation, internally/externally (also mucosa), intraperitoneally, subcutaneously, on/in internal organs (e.g., by endoscopy) in the form of tablets, liquid, gas, powder, injection, infusion, suppository, spray, ointments, plasters, as an antibiotic, cytostatic agent, microbicidal agent for the treatment of overweight (adenovirucide), rheumatism, dermatoses, gastritis, gastrointestinal diseases, bronchial diseases, depressions, arthritis, mucosal diseases, impotence, defective concentration, psychic disorders, migraine, sleep disturbance, i.e., vegetative symptoms, gastrointestinal symptoms, allergies and skin diseases, joint diseases, genital and hormonal disorders, infections, cancers and immune insufficiency. The required dose is dependent on the kind and severity of the disease, age, sex, weight and general health condition of the patient, and is usually within a range of from 0.1 to 10,000 mg, preferably from 0.5 to 1000 mg, per kg of body weight of the patient per day.

The present invention is further illustrated by means of the following Examples.

### EXAMPLES

Materials and Methods

All the following formulations are in percent by weight:
BHQ-1: 45% anisalcohol, 35% borneol, 20% rhodinol;
BHQ-2: 82% L-menthol, 8% anisol, 7% citronellol, 3% safrol;
BHQ-3: 98% propylene glycol, 2% allicin;
BHQ-4: 10% anisaldehyde, 90% glycerol;
BHQ-5: 25% cinnamyl alcohol, 25% linalool, 50% glycerol;
BHQ-6: 6% origanum, 8% coriander, 7% citric acid, 79% propylene glycol;
BHQ-A: 65% propylene glycol, 10% caffeic acid, 10% tannin, 5% resveratrol, 10% lactic acid;
BHQ-B: 1% benzyl alcohol, 88% propylene glycol, 1% tannin, 10% lactic acid;
BHQ-C: 70% propylene glycol, 20% anisalcohol, 5% quercetin, 5% tannin;
BHQ-AFC-1: 79% benzyl alcohol, 20% geraniol, 1% tannin;
BHQ-AFC-2: 95% benzyl alcohol, 5% mandelic acid;
BHQ-AFC-3: 70% benzyl alcohol, 30% catechol;
BHQ-MT: 5% cardamon, 5% anise, 10% Eucalyptus citriodora, 80% alginic acid;
BHQ-V: 20% eugenol, 50% citronellol, 20% citronellal, 10% ethyl cinnamate.

### Example 1

Microbicidal decontaminative effect: Flavoring-agent-containing medicaments BHQ 1-6 are fully effective (logRF3-5) against Gram-negative and Gram-positive bacteria, molds and yeasts (even toxin-forming ones) in both concentrated form and in aqueous (or other diluents) 1:10 (5%) dilution in accordance with suspension process according to DGHM Guidelines 2.3.1. The same is confirmed by the inhibition or inhibition halo test according to USP. The virus-inactivating effect of BHQ is confirmed by means of immunological HBsAg (antigen) test on an exemplary BHQ.

A: Quantative Suspension According to DGHM Guidelines 2.3.1

Product BHQ; test conditions: 20° C. without protein; medium: PBS and disinhibitor IV (3% Tween® 80+0.3% lecithin+3% saponin)

| Test | BHQ and conc. | | Time of action | | |
|---|---|---|---|---|---|
| strain (cfu/ml) | in volume % | | 60 min | Control | log RF |
| E. coli | 1 | 100 | 0 | 1,020,000 | |
| ATCC 11229 | | | 0 | | |
| (1.1 × 10⁹) | | | 0 | 1,020,000 | >5 |
| | 2 | 100 | 0 | 1,020,000 | |
| | | | 0 | | |
| | | | 0 | 1,020,000 | >5 |
| | 3 | 100 | 0 | 1,020,000 | |
| | | | 0 | | |
| | | | 0 | 1,020,000 | >5 |
| Ps. fluorescens | 1 | 100 | 0 | 4,500,000 | |
| DSM 6147 | | | 0 | | |
| (1.7 × 10⁹) | | | 0 | 4,500,000 | >5 |
| | 2 | 100 | 0 | 4,500,000 | |
| | | | 0 | | |
| | | | 0 | 4,500,000 | >5 |
| | 3 | 100 | 0 | 4,500,000 | |
| | | | 0 | | |
| | | | 0 | 4,500,000 | >5 |
| Staph. aureus | 1 | 100 | 0 | 1,360,000 | |
| ATCC 6638 | | | 0 | | |
| (1.9 × 10⁹) | | | 0 | 1,360,000 | >5 |
| | 2 | 100 | 0 | 1,360,000 | |
| | | | 0 | | |
| | | | 0 | 1,360,000 | >5 |
| | 3 | 100 | 0 | 1,360,000 | |
| | | | 0 | | |
| | | | 0 | 1,360,000 | >5 |
| Salm. enteritidis | 1 | 100 | 0 | 2,200,000 | |
| ATCC | | | 0 | | |
| (1.7 × 10⁹) | | | 0 | 2,200,000 | >5 |
| | 2 | 100 | 0 | 2,200,000 | |
| | | | 0 | | |
| | | | 0 | 2,200,000 | >5 |
| | 3 | 100 | 0 | 2,200,000 | |
| | | | 0 | | |
| | | | 0 | 2,200,000 | >5 |

B: Quantative Suspension Test According to DGHM Guidelines 2.3.1

Product BHQ; test conditions: 20° C. without protein; medium: PBS and disinhibitor IV (3% Tween® 80+0.3% lecithin+0.1% histidine+3% saponin)

| Test | BHQ and conc. | | Time of action | | |
|---|---|---|---|---|---|
| strain (cfu/ ml) | in volume % | | 60 min | Control | log RF |
| List. | 1 | 100 | 0 | 2,000,000 | |
| monocytogenes | | | 0 | | |
| ATCC 19111 | | | 0 | 2,000,000 | >5 |
| (1.3 × 10⁹) | 2 | 100 | 0 | 2,000,000 | |
| | | | 0 | | |
| | | | 0 | 2,000,000 | >5 |
| | 3 | 100 | 0 | 2,000,000 | |
| | | | 0 | | |
| | | | 0 | 2,000,000 | >5 |
| Candida albicans | 1 | 100 | 0 | 1,100,000 | |
| ATCC | | | 0 | | |
| (9.3 × 10⁹) | | | 0 | 1,100,000 | >5 |
| | 2 | 100 | 0 | 1,100,000 | |
| | | | 0 | | |
| | | | 0 | 1,100,000 | >5 |
| | 3 | 100 | 0 | 1,100,000 | |
| | | | 0 | | |
| | | | 0 | 1,00,000 | >5 |
| Ent. aerogenes | 1 | 100 | 0 | 4,400,000 | |
| ATCC 13048 | | | 0 | | |
| (7.0 × 10⁷) | | | 0 | 4,400,000 | >3 |
| | 2 | 100 | 0 | 4,400,000 | |
| | | | 0 | | |
| | | | 0 | 4,400,000 | >3 |
| | 3 | 100 | 0 | 4,400,000 | |
| | | | 0 | | |
| | | | 0 | 4,400,000 | >3 |
| Asp. niger | 1 | 100 | 0 | 580,000 | |
| ATCC | | | 0 | | |
| (3.2 × 10⁸) | | | 0 | 580,000 | >4.5 |
| | 2 | 100 | 0 | 580,000 | |
| | | | 0 | | |
| | | | 0 | 580,000 | >4.5 |
| | 3 | 100 | 0 | 580,000 | |
| | | | 0 | | |
| | | | 0 | 580,000 | >4.5 |

C: Quantative Suspension Test According to DGHM Guidelines 2.3.1

Product BHQ; test conditions: 20° C. without protein; medium: PBS and disinhibitor IV (3% Tween® 80+0.3% lecithin+0.1% histidine+3% saponin)

| Test | BHQ and conc. | | Time of action | | |
|---|---|---|---|---|---|
| strain (cfu/ml) | in volume % | | 60 min | Control | log RF |
| E. coli | 4 | 5 | 0 | 1,020,000 | |
| ATCC 11229 | | 1:20 | 0 | | |
| (1.1 × 10⁹) | | | 0 | 1,020,000 | >5 |
| | 5 | 5 | 0 | 1,020,000 | |
| | | 1:20 | 0 | | |
| | | | 0 | 1,020,000 | >5 |
| | 6 | 5 | 0 | 1,020,000 | |
| | | 1:20 | 0 | | |
| | | | 0 | 1,020,000 | >5 |
| Ps. fluorescens | 4 | 5 | 0 | 4,500,000 | |
| DSM 6147 | | 1:20 | 0 | | |
| (1.7 × 10⁹) | | | 0 | 4,500,000 | >5 |
| | 5 | 5 | 0 | 4,500,000 | |
| | | 1:20 | 0 | | |
| | | | 0 | 4,500,000 | >5 |
| | 6 | 5 | 0 | 4,500,000 | |
| | | 1:20 | 0 | | |
| | | | 0 | 4,500,000 | >5 |
| Staph. aureus | 4 | 5 | 0 | 1,360,000 | |
| ATCC 6538 | | 1:20 | 0 | | |
| (2.9 × 10⁹) | | | 0 | 1,360,000 | >5 |
| | 5 | 5 | 0 | 1,360,000 | |
| | | 1:20 | 0 | | |
| | | | 0 | 1,360,000 | >5 |
| | 6 | 5 | 0 | 1,360,000 | |
| | | 1:20 | 0 | | |
| | | | 0 | 1,360,000 | >5 |
| Salm. enteritidis | 4 | 5 | 0 | 2,200,000 | |
| ATCC | | 1:20 | 0 | | |
| (1.7 × 10⁹) | | | 0 | 2,200,000 | >5 |
| | 5 | 5 | 0 | 2,200,000 | |
| | | 1:20 | 0 | | |
| | | | 0 | 2,200,000 | >5 |
| | 6 | 5 | 0 | 2,200,000 | |
| | | 1:20 | 0 | | |
| | | | 0 | 2,200,000 | >5 |

D: Quantative Suspension Test According to DGHM Guidelines 2.3.1

Product BHQ; test conditions: 20° C. without protein; medium: PBS and disinhibitor IV (3% Tween® 80+0.3% lecithin+0.1% histidine+3% saponin)

| Test | BHQ and conc. | | Time of action | | |
|---|---|---|---|---|---|
| strain (cfu/ml) | in volume % | | 60 min | Control | log RF |
| List. | 4 | 5 | 0 | 2,000,000 | |
| monocytogenes | | 1:20 | 0 | | |
| ATCC 19111 | | | 0 | 2,000,000 | >5 |
| (1.3 × 10⁹) | 5 | 5 | 0 | 2,000,000 | |
| | | 1:20 | 0 | | |
| | | | 0 | 2,000,000 | >5 |
| | 6 | 5 | 0 | 2,000,000 | |
| | | 1:20 | 0 | | |
| | | | 0 | 2,000,000 | >5 |
| Candida albicans | 4 | 5 | 0 | 1,100,000 | |
| ATCC | | 1:20 | 0 | | |
| (9.3 × 10⁹) | | | 0 | 1,100,000 | >5 |
| | 5 | 5 | 0 | 1,100,000 | |
| | | 1:20 | 0 | | |
| | | | 0 | 1,100,000 | >5 |
| | 6 | 5 | 0 | 1,100,000 | |
| | | 1:20 | 0 | | |
| | | | 0 | 1,100,000 | >5 |
| Ent. aerogenes | 4 | 5 | 0 | 44,000 | |
| ATCC 13048 | | 1:20 | 0 | | |
| (7.0 × 10⁷) | | | 0 | 44,000 | >3 |
| | 5 | 5 | 0 | 44,000 | |
| | | 1:20 | 0 | | |
| | | | 0 | 44,000 | >3 |
| | 6 | 5 | 0 | 44,000 | |
| | | 1:20 | 0 | | |
| | | | 0 | 44,000 | >3 |
| Asp. niger | 4 | 5 | 0 | 580,000 | |
| ATCC | | 1:20 | 0 | | |
| (3.2 × 10⁸) | | | 0 | 580,000 | >4.5 |
| | 5 | 5 | 0 | 580,000 | |
| | | 1:20 | 0 | | |
| | | | 0 | 580,000 | >4.5 |
| | 6 | 5 | 0 | 580,000 | |
| | | 1:20 | 0 | | |
| | | | 0 | 580,000 | >4.5 |

### Example 2

Inhibition halo test; hole test: diameter of punchhole=8 mm; preliminary evaluation after 24 h

Diameter of inhibition halo in mm (evaluation 24 h)

| | BHQ-1 | BHQ-2 | BHQ-3 | BHQ-4 | BHQ-5 | BHQ-6 |
|---|---|---|---|---|---|---|
| E. coli | 16 | 28 | 24 | 38 | 19 | 19 |
| Ps. fluorescens | 18 | 24 | 26 | 36 | 44 | 17 |
| Staph. aureus | 20 | 33 | 18 | 28 | 30 | 35 |
| Salm. | 18 | 30 | 20 | 18 | 40 | 30 |
| enteritidis | | | | | | |
| L. monocyto- | 22 | 22 | 28 | 19 | 28 | 22 |
| genes | | | | | | |
| Candida | 20 | 2 | 22 | 40 | 41 | 35 |
| albicans | | | | | | |
| Asp. niger | 28 | 28 | 27 | 20 | 26 | 30 |
| Pen. commune | 18 | 32 | 30 | 26 | 23 | 40 |

### Example 3

Results of inhibition test, AFC variants; (the stated values are the averaged diameters of inhibition halo in cm)

| BHQ product/ | Weight per 3 | | | | | | | |
|---|---|---|---|---|---|---|---|---|
| variant | leaflets | Analyses No. | Strain A | Strain B | Strain C | Strain D | Strain E | Strain F |
| AFC 39 (1) | 0.0544 g | 2370 | 2.4 | 3.5 | 5.0 | >5 | 4.2 | >5 |
| AFC 39B (2) | 0.0654 g | 2371 | 2.5 | 3.2 | >5 | 4.1 | 4.6 | 4.8 |
| AFC 40 (3) | 0.0542 g | 2372 | 2.6 | 4.4 | 4.8 | 5.0 | 4.6 | >5 |
| AFC 40B | 0.0620 g | 2373 | 2.3 | 4.2 | 4.4 | 4.8 | 5.0 | >5 |
| AFC 41 | 0.0588 g | 2374 | 2.5 | 3.5 | 4.5 | 5.0 | 4.6 | 4.7 |
| AFC 41B | 0.0620 g | 2375 | 2.3 | 2.5 | 5.0 | 4.4 | 3.6 | 4.8 |
| AFC 42 | 0.0411 g | 2376 | 2.1 | 4.2 | 4.5 | 4.8 | 3.9 | >5 |
| AFC 42B | 0.0451 g | 2377 | 2.3 | 3.2 | 3.1 | 4.3 | 3.8 | 4.8 |
| AFC 5B | 0.0687 g | 2378 | 2.4 | 4.0 | 4.4 | >5 | 3.9 | >5 |
| AFC 5.0B | 0.0657 g | 2379 | 2.4 | 3.7 | >5 | 4.5 | 4.8 | >5 |
| 2B003 | 0.0495 g | 1262 | 2.2 | 4.0 | 5.0 | 5.0 | 4.4 | 5.0 |

Strain A: Geotrichum candidum (DSMZ 1240)
Strain B: Aspergillus niger (DSMZ 1988)
Strain C: Penicillium commune (DSMZ 2211)
Strain D: Penicillium roqueforti (DSMZ 1079)
Strain E: Aspergillus ochraceus (DSMZ 824)
Strain F: Fusarium chlamydosporum (DSMZ 62049)
2B003 - prior art “antimycotic agent”

### Example 4

A. Methods of Microbicidal, Decontaminative, Virucidal Agent Testing in HBsAg Inactivation Test: Hepatitis B//HTLV-HIV (AIDS)

The testing of the destruction of the immunological reactivity of HBsAg is effected in accordance with the “Richtlinien des Bundesgesundheitsamtes und der Deutschen Vereinigung zur Bekampfung der Viruskrankheiten zur Prüfung von mikrobiziden Mitteln auf Wirksamkeit gegen Viren” (Bundesgesundheitsbl. 25: 397 (1982)). The direct proof of infection is replaced by the detection of the destruction of HBsAg antigenicity.

The testing of BHQ agents was effected in a suspension experiment at 20 and 37° C. with and without additional protein loading. To 1 part of an HBsAg-containing serum (1:100 prediluted in PBS) were added 1 part of bidistilled water or 1 part of 2% serum albumin or 1 part of fetal calf serum, and 8 parts of the 1.25fold testing concentration of the BHQ agent.

After the end of the time of action, the activity of the agent was interrupted by a 1:100 dilution of the mixture with PBS containing ¹⁰% fetal calf serum. Then, each sample was examined for HBsAg in duplicate with a highest sensitivity solid-phase radio-immune test (Austria II, Abbott Diagnostics, Delkenheim). From both charges, a mean value of the bound radioactivity (cpm of ¹²⁵I-anti-HBs) was calculated.

As a starting value (100% value) for the calculation of the percent decrease of the binding of ¹²⁵I-anti HBs was used the mean value of quadruplicate experiments with the longest testing time used in the test to which 8 parts of bidistilled water had been admixed instead of 8 parts of the BHA agent. This mean value was 5391 cpm in the charge with bidistilled water, 4919 cpm in the charge with serum albumin and 4657 cpm in the charge with fetal calf serum.

As a zero percent value for the calculation of antigen inactivity was used the mean value of ten experiments of the agent concentration diluted 1:100 in PBS with 10% fetal calf serum. For 1% BHQ agent, this mean value was 216 cpm. Thus, it was within the range of the mean value of quadruplicate tests performed with the negative control serum (134 cpm) and the mean value of quadruplicate tests performed with using the PBS with 10% fetal calf serum as a diluent (211 cpm). Therefore, there is no activity of the BHQ agent on the HBsAg test system which would falsify the test result (“toxicity control”).

A complete inactivation of HBsAg was assumed when the cpm measured after the treatment with the BHQ agent was below 2.1 times the cpm of the negative control (i.e., less than 454 cpm). This corresponds to the limit of positivity stated by the manufacturer of the test. As a negative control, the above mean value of the ten experiments of the test concentration of the agent further diluted 1:100 in PBS with 10% fetal calf serum was used.

B. Effect on the Immunological Reactivity of HBsAg

1. Effect of 1% BHQ agent at 20° C.: Under the action of 1% BHQ agent, complete destruction of the immunological reactivity of HBsAg occurred at 20° C. even for a high protein load (experiment with fetal calf serum) after an action period of 24 h (Table 1). In the test, no binding of ¹²⁵I-anti-HBs beyond the limit of positivity could be measured.

2. Effect of 1% BHQ agent at 37° C.: Also under the action of 1% BHQ agent at 37° C., HBsAg was completely destroyed after a period of action of 24 h independently of the protein load (Table 2).

C. Evaluation of HBV-Inactivating Effect

Due to the selected test criteria, a microbicidal agent is attested an HBC-inactivating effect in the antigen inactivation test if a complete destruction of the immunological reactivity of HBsAg occurred under the action of such an agent. Independently of the protein load, this is the case with 1% BHQ agent both at 20° C. and at 37° C. after a period of action of 24 h.

This is an excellent test result. Comparative examinations have shown that the hurdle to be taken by a microbicidal agent in the antigen-inactivation test is extraordinarily high. In the testing of other microbicidal agents, Thraenhart and Kuwert found residual amounts of HBsAg in an antigen-inactivation test under conditions which showed a good HBV activity with MADT (s. l.).

This statement appears to be justified also because HBV is substantially less resistant than has been assumed to date. Although the virucidal effect of alcoholic microbicidal agents is considered a limited one, it could be shown that 10⁶ HBV doses which are infectious towards chimpanzees was inactivated by 70% isopropyl alcohol for 10 min at room temperature. In this experiment, there were complicated conditions of action for the microbicidal agent because the infectious serum was dried onto a plastic surface (Bond et al., J. Clin. Microbiol. 18: 535 (1983)). Under these conditions, the same working group could also show complete inactivation of HBV with an iodine-containing detergent in a chimpanzee test.

Therefore, considering all the facts, an excellent HBV-inactivating effect can be attested to 1% BHQ agent after a period of action of 24 h both at 20° C. and at 37° C. It appears to be particularly important that this high effectiveness is also present when the protein load is high.

Recently, there was repeatedly asked the question of whether the microbicidal agents successfully checked according to the Guidelines of the Bundesgesund-heitsamt and the DW are also effective towards the pathogen of AIDS (acquired immunodeficiency syndrome). This can be answered to the positive without reservation because the HTLV-III/LAV/HIV virus is one of the most sensitive viral pathogens known. Heating at 56° C. for 30 minutes already inactivates the virus (Spire et al., Lancet, I: 188-189 (1985)). Also, the virus is quickly inactivated at pH values of below 7 and above 10. The action of pH 5.7 for 10 min already reduces the viral concentration to one thousandth of the initial quantity (Martin et al., J. Infect. Dis. 152: 400-403 (1985)). Therefore, a separate test of microbicidal agents for effectiveness against HTLV-III/LAV/HIV does not appear necessary when the test against the highly resistant test viruses (polio, adenovirus, papova and pox viruses) was successful.

The test of a microbicidal agent for effectiveness against HBV is also a good basis for drawing conclusions on effectiveness against HTLV-III/LAV/HIV. Both viruses have a lipoprotein envelope and also share many other structural and biological similarities. After the action of as little as 19% ethanol, the viral enzyme reverse transcriptase, which is necessary for HTLV-III/LAV/HIV proliferation, can no longer be detected (Spire et al., Lancet, II: 899-901 (1984)). The action of 50% ethanol at 23° C. for 10 min destroys the infectiousness of the virus with and without additional protein loading (Piszzkiewicz et al., Lancet II: 1188-1189 (1985)). Presumably, the mild lipid solvent ethanol acts via a destruction of the lipoprotein envelope of the virus. The AIDS proofness of clotting factor preparations precipitated with cold 20% ethanol (period of action of 10 h) could be proven clinically (Gazengel, Larrieu, Lancet, II: 1189 (1985)).

Since the microbicidal agent BHQ-V tested here has an excellent HBV-destroying effect, it can be considered that the less stable HTLV-III/LAV/HIV will also be inactivated with certainty under the same conditions.

To one part of HBsAg-containing serum, there were added 1 part of bidistilled water or 1 part of 2% serum albumin or 1 part of fetal calf serum and 8 parts of 1.25 times the testing concentration of the BHQ agent.

TABLE 1
Effect at 20° C. of 1% BHQ-V on antigenicity of HBsAg

| | cpm in HBsAg test after the | | | | | |
|---|---|---|---|---|---|---|
| | end of the time of action | | | | | |
| | with one part bidist. | | with one part of 2% | | with one part of | |
| Time of action (hours) | water | | serum albumin | | fetal calf serum | |
| Antigen control without | 5391 | (100%) | 4919 | (100%) | 4657 | (100%) |
| microbicidal agent | | | | | | |
| 24 | 163 | negative | 140 | negative | 145 | negative |
| 48 | 104 | negative | 154 | negative | 148 | negative |
| Microbicidal agent | | | 216 | (0%)* | | |
| without HBsAg | | | | | | |

TABLE 2
Effect at 37° C. of 1% BHQ-V on antigenicity of HBsAg

| | cpm in | | | | | |
|---|---|---|---|---|---|---|
| | HBsAg test after the end of the time of action | | | | | |
| | | | with one part | | with one | |
| | with one part | | of 2% serum | | part of fetal | |
| Time of action (hours) | bidist. water | | albumin | | calf serum | |
| Antigen control | 5391 | (100%) | 4919 | (100%) | 4657 | (100%) |
| without microbicidal | | | | | | |
| agent | | | | | | |
| 24 | 144 | negative | 166 | negative | 147 | negative |
| 48 | 161 | negative | 148 | negative | 150 | negative |
| Microbicidal agent | | | 216 | (0%)* | | |
| without HBsAg | | | | | | |

*The detection limit of HBsAg is 2.1 times the cpm of the negative control (here: 454 cpm) in the Austria II test according to the indications given by the manufacturer.

### Example 5

Decontaminative Antitoxin Action (e.g. Mycotoxin, e.g., Aflatoxin from Aspergillus parasiticus) of the Medicament Containing the Flavoring Agent

BHQ (AFC and MT) was tested in vitro in 0.2% and 0.4% application for their reactivity against toxins from microorganisms. Thus, an in-vitro support which contains surface structures which are similarly difficult to access as those of the human or animal intestine was desired.

Aspergillus parasiticus as a reference fungus with aflatoxin cleavage was grown on the support (raw coffee). In contrast to the zero sample, the support was treated with BHQ by spraying. The aflatoxin content was measured by means of the Mycotoxin Testing System VICAM (Fluorometric and HPLC Method) Aflatest®. The Aflatest® is effected according to the following scheme:

Collection of sample
Milling and weighing of the sample
Adding salt and a mixture of methanol/water to the sample
Filtering
↓
Dilution and filtering
Diluting a portion of the collected sample with water
Filtering
↓
Affinity chromatography
Charging the sample onto an affinity column
Washing the column with water
Eluting the aflatoxins from the column with methanol
Collecting the eluate in a cuvette
↓
Aflatoxin measurement
Injecting into HPLC
or
Adding developer to the eluate
Inserting the cuvette into a fluorimeter
Storing the digital readout after 1 min

Since aflatoxins, like many toxins from microorganisms, have clearly cancerogenic properties, as described in Samson et al., Introduction to Food Borne Fungi”, and Mücke et al., “Schimmelpilze” (1999), the toxin-reducing effect, e.g., up to 42% in experiments with BHQ shown here, is of high importance.

Examination Methods:

Yeasts and molds (Section 35 LMBG * * * L01.00-37, December. 91, mod.)

Number of germs (Section 35 LMBG * * * L01.00-5, November. 93, mod.)

Aflatoxin and ochratoxin test Mycotoxin Testing System VICAM (Fluorometric & HPLC Method)

| | | | | **Number | Aflatoxin | Aflatoxin, |
|---|---|---|---|---|---|---|
| | | Molds/g, | Yeasts/g, | of germs/g, | [ppb], day | reduction |
| Analysis | Designation | day 30 of | day 30 of | day 30 of | 30 of | in % by |
| No. | of product | examination | examination | examination | examination | BHQ |
| 590 | sterile control | *1.4 × 10⁷ | <10 | 1.0 × 10⁴ | 1.5 | |
| 592 | zero sample | 1.2 × 10⁷ | <10 | 9.0 × 10⁶ | 11 | 0% |
| | parasiticus | | | | | |
| 595 | parasiticus | 2.5 × 10⁴ | <10 | 2.2 × 10⁴ | 8.8 | 20% |
| | AFC/0.2 | | | | | |
| 596 | parasiticus | 1.4 × 10⁴ | <10 | 1.6 × 10⁴ | 6.4 | 42% |
| | AFC/0.4 | | | | | |
| 599 | parasiticus MT | 4.0 × 10⁴ | <10 | 7.5 × 10⁴ | 6.4 | 42% |
| | 10/0.2 | | | | | |
| 600 | parasiticus MT | 1.6 × 10⁴ | <10 | 1.8 × 10⁴ | 6.4 | 42% |
| | 10/0.4 | | | | | |

**exclusively the molds used for contamination: Aspergillus parasiticus
*Mb = VDLUFA-Methodenbuch Vol. VI, 4th Ed. 1985, and further supplements, VDLUFA-Verlag Darmstadt, E. Lechner (Ed.)
***Section 35 LMBG = Lebensmittel- und Bedarfsgegenständegesetz [German Food and Consumer Goods Act]; official collection of examination methods according to Section 35

### Example 6

Examination for the possible formation of selections (shift of germ spectrum) or resistance in practical long-term application (model: agent containing flavoring agents) (final differentiation of the germ flora (bacteria/molds) in, e.g., cheese maturing rooms, with and without treatment of the air with BHQ agent (mixture of flavoring agents))

Examination: RCS air-borne germ strip TC, total germ number; VM yeasts and molds

Begin of examination: 5 months after BHQ application

Collection of sample: Place: Cheese store of the cheese factory, High Flow air-borne germ collecting device, supplied by Biotest

For testing a medicament containing flavoring agents for its selection behavior or resistance formation during its decontaminative activity without experiments on humans (phases I-III), a five-month experiment of application in practice was performed in air with 5 ppb/m³/h in a cheese-maturing room, since BHQ is also capable of disinfecting air.

Coated cheeses (e.g., Gouda) are in part treated with high antimycotic (natamycin) dosages lest mold should grow thereon during and after the maturing. A maturing period has a duration of about 4 weeks. The air present in the maturing rooms is likely to have already produced resistant microorganisms due to antimycotic treatment employed for years. Thus, the bacterium Listeria, for example, is found increasingly in/on cheese, which has already resulted in considerable health risks. It is all the more astonishing that a germ reduction of >90% occurs already at a dosage of as low as 5 ppb of BHQ.

At sublethal dosages (underdosing), antibiotics produce resistances. It is to be assumed that BHQ was used at too low a dosage (sublethal) in the application experiment, and yet there was no visible resistance or shift of spectrum after more than five months of application, so that BHQ can be considered non-resistance-forming. Also, due to the structure of the flavoring agents, no resistances have been produced evidently for millenniums, and due to the variability of the flavoring agents, it also appears hardly possible that microorganisms can identify them immediately as “enemies”, of which the microorganisms are evidently capable with “monosubstances or their mixtures” (such as anti-infective agents and the like). This also means that resistances mostly occur, for example, with anti-infective agents of the prior art at sublethal doses (underdosing).

1. Germ Content (Colony-Forming Units Per 1000 Liters of Air)

| Measuring sites | Number of germs | Yeasts | Molds |
|---|---|---|---|
| Maturing room R7 without BHQ | | 0 | 1443 |
| Maturing room R7 without BHQ | 103 | | |
| Maturing room R13 without BHQ | | 0 | 2482 |
| Maturing room R13 without BHQ | 420 | | |
| Maturing room R14 with BHQ | | 0 | 191 |
| Maturing room R14 with BHQ | 29 | | |

2. Analysis of Flora

| | Maturing room | Maturing room | Maturing room |
|---|---|---|---|
| | R7 without BHQ | R13 without BHQ | R14 with BHQ |
| Bacteria | | | |
| Bacillus subtilis | + | + | + |
| Staphylococcus | + | + | + |
| epidermidis | | | |
| Germ from the | + | + | + |
| group of | | | |
| coryneform | | | |
| bacteria | | | |
| Flavobacterium | + | + | + |
| sp. | | | |
| Molds | | | |
| Aspergillus | + | + | + |
| versicolor | | | |
| Penicillium sp. 1 | + | + | + |
| (corylophilum) | | | |
| Penicillium sp. 2 | + | + | + |
| (glabrum) | | | |
| Penicillium sp. 3 | + | + | + |
| Cladosporium | + | + | + |
| cladosporioides | | | |

+ detectable

The microbiological examinations of the ambient air exhibit a clear reduction of the germ content in the germ range of the room fogged with BHQ similarly for both bacteria and molds.

An evaluation of the analysis of flora performed here makes it clear that in principle the same types of germs were found in the fog room as in the unfogged cheese store. All air-borne germs isolated from the fog room were also found in the untreated air. Thus, germ selection due to the application of BHQ could not be detected. Due to the strong reduction in number of bacteria and molds, it is to be considered that no formation of selections and resistances has occurred after five months of permanent BHQ application.

### Example 7

A. Regenerative effect of the medicament containing flavoring agents: During the development works and bacteriological tests, it was noticed time and again that, in contrast to microbicidal agents of the prior art, the synergies of the flavoring agents not only have protagonistic decontaminative microbicidal properties, but are also capable of doing the very opposite, namely display antagonistic regenerative properties, i.e., stabilize or preserve or even promote the growth of microorganisms.

In all kinds of human and animal application forms of medical and pharmaceutical types, this would have the advantage that either the “positive” microorganisms necessary to life in/on the body would be preserved, or their growth would be reanimated or enhanced. Also, microorganisms (e.g., lactobacilli, Bac. subtilis, positive coliform bacteria and the like) can be regeneratively supplied to the body and then preserved, stabilized or enhanced in their body cell adaptation and in their function and proliferated, so that a regenerative effect necessary to the body is possible. Given about 40 trillions of body cells, but only 100 trillions of microorganisms (about 400 to 500 species), this is an altogether plausible and necessary process, all the more so since the equilibrium of the necessary species of microorganisms present in the body and their quantity and quality (resistances or selectionism, i.e., other germs overgrow or increase) in many humans and animals is already disturbed due to the way of living and the medication of recent times, which is the cause of many clinical pictures as described above.

The “American Journal of Gastroenterology” and the scientific journal “The Lancet” report, inter alia, more and more about these phenomena since we have been able to find also biotechnological answers to questions. Thus, it can be conceived that a multi-step application method or a combined application or individual application of the respective “medicaments containing flavoring agents” can be employed. The dosage forms in which they are employed can be chosen from the following:
oral (tablets, liquid, powder, gas phase (mist))
intravenous, intramuscular (injections, infusions, liquid, powder or the like)
rectal (suppository or the like)
inhalational (liquid, gas phase, spray)
contra preparation (application on mucosa: internal, liquid, powder, gas phase or the like; external application: ointment, liquid, powder, gas phase or the like)
intraperitoneal (i.p.) (liquid, solid, gas phase)
subcutaneous (s.c.) (liquid, solid, gas phase)
on/in internal organs (injection, infusion, by endoscopy or the like)
   B. Application Methods
   I. Multi-Step:

1. “decontaminative agent” for disinfecting pathogens or infective agents in/on the body with medicaments containing flavoring agents (depending on the formulation and dosage)

2. “regenerative agent” for regenerating (preservation or promotion of growth) necessary microorganisms in/on the body with medicaments containing flavoring agents (depending on the formulation and dosage)

II. Combined Methods:

1. +2. simultaneously

1. +2. successively

III. Individual Application: 1., 2. and/or Combined or Multi-Step with Medicaments of the Prior Art (e.g., Anti-Infective Agents, Antidepressants, Cytostatic Agents, Contraceptives and Many More).

C. Ouantitative suspension experiments: According to the quantitative suspension method in accordance with DBHM Guidelines 2.3.1 and the inhibitor/inhibition halo test (USP), the regenerative, bacteriological, antagonistic properties of the synergies of the flavoring agents in the medicament containing flavoring agents according to the invention (BHQ) are demonstrated.

C.I. Quantitative Suspension Experiment According to DGHM Guidelines 2.3.1

Product BHQ; test conditions: 20° C. without protein; medium: PBS and disinhibitor IV (3% Tween® 80+0.3% lecithin+0.1% histidine+3% saponin)

| | BHQ and | | | | |
|---|---|---|---|---|---|
| | conc. in | | Time of action | | D > |
| Test strain (cfu/ml) | volume % | | 60 min | Control | growth |
| Lactobacillus | A | 0.01 | 130,000 | 65,000 | + |
| acidophilus | A | 0.05 | 145,000 | 65,000 | + |
| (3.1 × 10⁷) | A | 0.5 | 95,000 | 65,000 | + |
| Lactobacillus bifidus | A | 0.01 | 1,850,000 | 1,140,000 | + |
| (7.0 × 10⁸) | A | 0.05 | 2,600,000 | 1,140,000 | + |
| | A | 0.5 | 2,220,000 | 1,140,000 | + |
| Bacillus subtilis | A | 0.01 | 210,000 | 84,000 | + |
| (6.2 × 10⁷) | A | 0.05 | 170,000 | 84,000 | + |
| | A | 0.5 | 110,000 | 84,000 | + |
| Coliform bacteria | A | 0.01 | 1,040,000 | 950,000 | + |
| (8.9 × 10⁸) | A | 0.05 | 1,300,000 | 950,000 | + |
| | A | 0.5 | 1,580,000 | 950,000 | + |

C.II. Quantitative Suspension Experiment According to DGHM Guidelines 2.3.1

Product BHQ; test conditions: 20° C. without protein; medium: PBS and disinhibitor IV (3% Tween® 80+0.3% lecithin+0.10% histidine+30% saponin)

| | BHQ and | | Time | | |
|---|---|---|---|---|---|
| | conc. in | | of action | | |
| Test strain (cfu/ml) | volume % | | 6 hours | Control | D > growth |
| Lactobacillus | A | 0.01 | 530,000 | 65,000 | + |
| acidophilus | A | 0.05 | 385,000 | 65,000 | + |
| (3.1 × 10⁷) | A | 0.5 | 410,000 | 65,000 | + |
| Lactobacillus | A | 0.01 | 10,100,000 | 1,140,000 | + |
| bifidus | A | 0.05 | 8,500,000 | 1,140,000 | + |
| (7.0 × 10⁸) | A | 0.5 | 9,900,000 | 1,140,000 | + |
| Bacillus subtilis | A | 0.01 | 900,000 | 84,000 | + |
| (6.2 × 10⁷) | A | 0.05 | 650,000 | 84,000 | + |
| | A | 0.5 | 720,000 | 84,000 | + |
| Coliform bacteria | A | 0.01 | 14,000,000 | 950,000 | + |
| (8.9 × 10⁸) | A | 0.05 | 7,200,000 | 950,000 | + |
| | A | 0.5 | 1,350,000 | 950,000 | + |

C.III. Quantitative Suspension Experiment According to DGHM Guidelines 2.3.1

Product BHQ; test conditions: 20° C. without protein; medium: PBS and disinhibitor IV (3% Tween® 80+0.30% lecithin+0.1% histidine+30% saponin)

| | BHQ and | | Time | | |
|---|---|---|---|---|---|
| | conc. in | | of action | | |
| Test strain (cfu/ml) | volume % | | 60 min | Control | D > growth |
| Lactobacillus | B | 0.01 | 73,000 | 65,000 | + |
| acidophilus | B | 0.05 | 85,000 | 65,000 | + |
| (3.1 × 10⁷) | B | 0.5 | 98,000 | 65,000 | + |
| Lactobacillus | B | 0.01 | 1,400,000 | 1,140,000 | + |
| bifidus | B | 0.05 | 1,210,000 | 1,140,000 | + |
| (7.0 × 10⁸) | B | 0.5 | 1,320,000 | 1,140,000 | + |
| Bacillus subtilis | B | 0.01 | 92,000 | 84,000 | + |
| (6.2 × 10⁷) | B | 0.05 | 120,000 | 84,000 | + |
| | B | 0.5 | 98,000 | 84,000 | + |
| Coliform bacteria | B | 0.01 | 1,040,000 | 950,000 | + |
| (8.9 × 10⁸) | B | 0.05 | 1,400,000 | 950,000 | + |
| | B | 0.5 | 1,670,000 | 950,000 | + |

C.IV. Quantitative Suspension Experiment According to DGHM Guidelines 2.3.1

Product BHQ; test conditions: 20° C. without protein; medium: PBS and disinhibitor IV (3% Tween® 80+0.3% lecithin+0.1% histidine+30% saponin)

| Test | BHQ and conc. | | Time of action | | D > |
|---|---|---|---|---|---|
| strain (cfu/ml) | in volume % | | 6 hours | Control | growth |
| Lactobacillus | B | 0.01 | 180,000 | 65,000 | + |
| acidophilus | B | 0.05 | 165,000 | 65,000 | + |
| (3.1 × 10⁷) | B | 0.5 | 130,000 | 65,000 | + |
| Lactobacillus | B | 0.01 | 3,500,000 | 1,140,000 | + |
| bifidus | B | 0.05 | 2,300,000 | 1,140,000 | + |
| (7.0 × 10⁸) | B | 0.5 | 2,750,000 | 1,140,000 | + |
| Bacillus | B | 0.01 | 220,000 | 84,000 | + |
| subtilis | B | 0.05 | 195,000 | 84,000 | + |
| (6.2 × 10⁷) | B | 0.5 | 250,000 | 84,000 | + |
| Coliform | B | 0.01 | 1,800,000 | 950,000 | + |
| bacteria | B | 0.05 | 2,550,000 | 950,000 | + |
| (8.9 × 10⁸) | B | 0.5 | 2,200,000 | 950,000 | + |

C.V. Ouantitative Suspension Experiment According to DGHM Guidelines 2.3.1

Product BHQ; test conditions: 20° C. without protein; medium: PBS and disinhibitor IV (3% Tween® 80+0.3% lecithin+0.1% histidine+3% saponin)

| Test | BHQ and conc. | | Time of action | | D > |
|---|---|---|---|---|---|
| strain (cfu/ml) | in volume % | | 60 min | Control | growth |
| Lactobacillus | C | 0.01 | 71,000 | 65,000 | + |
| acidophilus | C | 0.05 | 70,000 | 65,000 | + |
| (3.1 × 10⁷) | C | 0.5 | 76,000 | 65,000 | + |
| Lactobacillus | C | 0.01 | 1,200,000 | 1,140,000 | + |
| bifidus | C | 0.05 | 1,190,000 | 1,140,000 | + |
| (7.0 × 10⁸) | C | 0.5 | 1,220,000 | 1,140,000 | + |
| Bacillus subtilis | C | 0.01 | 95,000 | 84,000 | + |
| (6.2 × 10⁷) | C | 0.05 | 90,000 | 84,000 | + |
| | C | 0.5 | 110,000 | 84,000 | + |
| Coliform bacteria | C | 0.01 | 1,030,000 | 950,000 | + |
| (8.9 × 10⁸) | C | 0.05 | 1,200,000 | 950,000 | + |
| | C | 0.5 | 990,000 | 950,000 | + |

C.VI. Quantitative Suspension Experiment According to DGHM Guidelines 2.3.1

Product BHQ; test conditions: 20° C. without protein; medium: PBS and disinhibitor IV (3% Tween® 80+0.3% lecithin+0.1% histidine+3% saponin)

| Test | BHQ and conc. | | Time of action | | D > |
|---|---|---|---|---|---|
| strain (cfu/ml) | in volume % | | 60 min | Control | growth |
| Lactobacillus | C | 0.01 | 90,000 | 65,000 | + |
| acidophilus | C | 0.05 | 95,000 | 65,000 | + |
| (3.1 × 10⁷) | C | 0.5 | 103,000 | 65,000 | + |
| Lactobacillus | C | 0.01 | 1,420,000 | 1,140,000 | + |
| bifidus | C | 0.05 | 1,310,000 | 1,140,000 | + |
| (7.0 × 10⁸) | C | 0.5 | 1,300,000 | 1,140,000 | + |
| Bacillus subtilis | C | 0.01 | 115,000 | 84,000 | + |
| (6.2 × 10⁷) | C | 0.05 | 121,000 | 84,000 | + |
| | C | 0.5 | 133,000 | 84,000 | + |
| Coliform bacteria | C | 0.01 | 1,200,000 | 950,000 | + |
| (8.9 × 10⁸) | C | 0.05 | 1,360,000 | 950,000 | + |
| | C | 0.5 | 1,280,000 | 950,000 | + |

### Example 8

Inhibition halo test; hole test: diameter of punchhole=8 mm; preliminary evaluation after 24 h

Diameter of inhibition halo in mm (evaluation 24 h)

| | BHQ A | BHQ B | BHQ C |
|---|---|---|---|
| Lact. acidophilus | 0 | 0 | 0 |
| Lact. bifidus | 0 | 0 | 0 |
| Lact. bulgaris | 0 | 0 | 0 |
| Lact. casei | 0 | 0 | 0 |
| Bac. subtilis | 0 | 0 | 0 |
| Coliform bacteria | 0 | 0 | 0 |
| Enterococci | 2 | 0 | 0 |

1. A medicament comprising a microbicidal composition, the microbial composition comprising:
(I) one or more generally recognized as safe (GRAS) flavor alcohols (a) selected from the group consisting of benzyl alcohol, acetoin, propyl alcohol, iso-propyl alcohol, propylene glycol, glycerol, n-butyl alcohol, iso-butyl alcohol, hexyl alcohol, L-menthol, octyl alcohol, cinnamyl alcohol, α-methylbenzyl alcohol, heptyl alcohol, n-amyl alcohol, iso-amyl alcohol, anisalcohol, citronellol, n-decyl alcohol, geraniol, β-γ-hexanol, lauryl alcohol, linalool, nerolidol, nonadienol, nonyl alcohol, rhodinol, terpineol, borneol, clineol, anisole, cuminyl alcohol, 10-undecene-1-ol, 1-hexadecanol and derivatives thereof; and
(II) one or more flavoring agents selected from the group consisting of:
(II-1) polyphenol compounds (b) or their derivatives; and
(II-2) GRAS flavor acids (c) or their derivatives.

2. The medicament according to claim 1, wherein:
(II-1) said polyphenol compounds (b) are selected from the group consisting of catechol, resorcinol, hydroquinone, phloroglucinol, pyrogallol, cyclohexane, usnic acid, acylpolyphenols, lignins, anthocyans, flavones, catechols, gallic acid derivatives, caffeic acid, flavonoids, derivatives of the mentioned polyphenols, and extracts from Camellia, Primula; and
(II-2) said GRAS flavor acids (c) are selected from the group consisting of acetic acid, aconitic acid, adipic acid, formic acid, malic acid, capronic acid, hydrocinnamic acid, pelargonic acid, lactic acid, phenoxyacetic acid, phenylacetic acid, valeric acid, iso-valeric acid, cinnamic acid, citric acid, mandelic acid, tartaric acid, fumaric acid, tannic acids and derivatives.

3. The medicament according to claim 1, wherein said microbicidal composition comprises:
from 0.1 to 99% by weight of component (I);
from 0 to 25% by weight of component (II-1); and
from 0 to 70% by weight of component (II-2).

4. The medicament according to claim 3, wherein said microbicidal composition comprises (I-1) benzyl alcohol.

5. The medicament according to claim 3, wherein said microbicidal composition further comprises a GRAS flavoring agents selected from the group consisting of (d) phenols, (e) esters, (f) terpenes, (g) acetals, (h) aldehydes and (i) essential oils.

6. The medicament according to claim 5, wherein said GRAS flavoring agents are selected from the group consisting of phenols (d) and essential oils (i).

7. The medicament according to claim 4, wherein said microbicidal composition contains one or two further GRAS flavor alcohols (a) and at least one polyphenol compound (b).

8. The medicament according to claim 7, wherein said polyphenol compound (b1) is tannin.

9. The medicament according to claim 1, wherein said microbicidal composition contains at least two GRAS essential oils (i).

10. The medicament according to claim 9, wherein said microbicidal composition further comprises at least one GRAS flavoring agents selected from alcohols (a), polyphenol compounds (b), acids (c), phenols (d), esters (e), terpenes (f), acetals (g), aldehydes (h), derivatives thereof, and flavor carrier agents (j).

11. The medicament according to claim 10, wherein said flavor carrier agents (j) are selected from the group consisting of GRAS flavoring agents and non-GRAS compounds having flavor carrier properties.

12. The medicament according to claim 10, wherein said microbicidal composition contains:
from 0.01 to 20% by weight of GRAS essential oils (i);
from 0 to 80% by weight of GRAS flavoring agents (a) to (h); and
from 0 to 80% by weight of flavor carrier agents (j).

13. The medicament according to claim 10, wherein said microbicidal composition contains at least three GRAS essential oils (i) or said further GRAS flavoring agents are selected from the group consisting of anisole and quercitin.

14. The medicament according to claim 1, wherein said microbicidal composition contains a an aromatic GRAS flavor alcohol (a).

15. The medicament according to claim 14, wherein said microbicidal composition contains less than 50% by weight of ethanol, isopropanol, or benzyl alcohol, combinations thereof.

16. The medicament according to claim 14, wherein said microbicidal composition contains at least one of a hydrophilic alcoholic GRAS flavoring agent and a hydrophilic non-alcoholic GRAS flavoring agent.

17. The medicament according to claim 1, wherein said microbicidal composition contains at least one lipophilic GRAS (generally recognized as safe) flavoring agent and at least one hydrophilic GRAS flavoring agent.

18. The medicament according to claim 17, wherein said lipophilic GRAS flavoring agents are selected from the group consisting of (aₗ) lipophilic GRAS flavor alcohols or their derivatives, (b) polyphenol compounds, (cₗ) lipophilic GRAS flavor acids or their derivatives, (d) phenols or their derivatives, (eₗ) lipophilic esters, (f) terpenes, (g) acetals, (hₗ) lipophilic aldehydes and (i) essential oils.

19. The medicament according to claim 18, wherein said lipophilic GRAS flavor alcohols are selected from the group consisting of aromatic GRAS flavor alcohols, aliphatic GRAS flavor alcohols, and derivatives thereof.

20. The medicament according to claim 17, wherein:
(I) said hydrophilic GRAS flavoring agent is selected from the group consisting of a hydrophilic alcoholic GRAS flavoring agent (aₕ) and a hydrophilic non-alcoholic GRAS flavoring agent; and
(II) said hydrophilic non-alcoholic GRAS flavoring agents is selected from the group consisting of a hydrophilic organic GRAS flavor acid (cₕ) having from 1 to 15 carbon atoms or a physiologically acceptable salt thereof, a hydrophilic acetate (eₕ), and a hydrophilic aldehyde (hₕ).

21. The medicament according to claim 20, wherein said microbicidal composition contains less than 50% by weight
of benzyl alcohol or of a mixture of benzyl alcohol with ethanol and/or isopropanol; or
benzyl alcohol and/or a polyphenol compound (b), but no further GRAS flavor alcohols.

22. The medicament according to claim 20, wherein said microbicidal composition contains two lipophilic GRAS flavor alcohols (aₗ), but no benzyl alcohol and no polyphenol compounds (b).

23. The medicament according to claim 21, wherein said microbicidal composition contains exclusively non-alcoholic hydrophilic GRAS flavoring agents.

24. The medicament according to claim 17, wherein said microbicidal composition contains:
(III) one or more GRAS flavor alcohols (a) or their derivatives; and
(IV) one or more flavoring agents selected from polyphenol compounds (component IV-1) and lipophilic GRAS flavor acids (c) or their derivatives (component IV-2).

25. The medicament according to claim 24, wherein said microbicidal composition further comprises at least one GRAS flavoring agents selected from the group consisting of (d) phenols or their derivatives, (eₗ) lipophilic esters, (f) terpenes, (g) acetals, (hₗ) lipophilic aldehydes and (i) essential oils.

26. The medicament according to claim 24, wherein said lipophilic GRAS acid (c) is selected from the group consisting of adipic acid, capronic acid, pelargonic acid, phenoxyacetic acid, valeric acid, iso-valeric acid, cinnamic acid, mandelic acid, and derivatives thereof.

27. The medicament according to claim 24, wherein component (III) of said microbicidal composition contains benzyl alcohol.

28. The medicament according to claim 27, wherein component (IV) of said microbicidal composition contains one or more polyphenol compounds (b).

29. The medicament according to claim 27, wherein said microbicidal composition contains further comprises lipophilic GRAS flavoring agents (d) to (i).

30. The medicament according to claim 27, wherein said microbicidal composition comprises one or two lipophilic GRAS flavor alcohols (aₗ) and at least one polyphenol compound (b).

31. The medicament according to claim 1, wherein said microbicidal composition contains at least one GRAS flavoring agent having one or more double bonds (Δ).

32. (Canceled)

33. The medicament according to claim 31, wherein said composition further comprises at least one of essential oils (i) and hydrophilic GRAS flavor agents.

34. The medicament according to claim 1, wherein said microbicidal composition further comprises at least one of monovalent or polyvalent alcohols having from 2 to 10 carbon atoms, emulsifiers, stabilizers, antioxidants, preservatives, solvents, and carriers (j).

35. The medicament according to claim 1, wherein said microbicidal composition does not contain any derivatives of said GRAS flavoring agents and consists of GRAS flavoring agents.

36. The medicament according to claim 1, which is a decontaminative agent, a regenerative agent, a virucide, an agent for toxin reduction, and combinations thereof.

37. The medicament according to claim 1, wherein said medicament is in a form adapted for a route of administration selected from the group consisting of inhalation, oral, intravenous, intramuscular, rectal, contact preparation, internal/external intraperitoneal, subcutaneous, on/in internal organs and is in a form selected from the group consisting of a tablets, a liquid, a gas, a powder, an injection, an infusion, a suppository, a spray, an ointments and a plaster.

38-40. (Canceled)

41. A food supplement or animal feed comprising the microbicidal composition of claim 1.

42. A method for the treatment of infectious diseases and respiratory diseases in one of humans and animals, the method comprising administering the microbicidal composition of claim 1 to one of humans and animals in need of such a treatment.

43. The medicament according to claim 4, further comprising at least one of:
(I-2) a GRAS flavor alcohol (a) or a derivative thereof;
(II-1) a polyphenol compound (b) or a derivative thereof; and
(II-2) a GRAS acid (c) or a derivative thereof.

44. The medicament according to claim 43, wherein said microbial composition comprises:
from 0.1 to 99% by weight of benzyl alcohol;
from 0 to 99.8% by weight of component (I-2); and
from 0 to 25% by weight of component (II-1); and
from 0 to 70% by weight of component (II-2).

45. The medicament of claim 5, wherein the GRAS phenol is selected from the group consisting of thymol, methyleugenol, acetyleugenol, safrol, eugenol, isoeugenol, anethole, phenol, methylchavicol, carvacrol, α-bisabolol, fornesol, anisole, propenylguaethol, and derivatives thereof.

46. The medicament of claim 5, wherein the GRAS ester is selected from the group consisting of iso-amyl acetate, benzyl acetate, benzylphenyl acetate, n-butyl acetate, cinnamyl acetate, citronellyl acetate, ethyl acetate, eugenol acetate, geranyl acetate, hexyl acetate, hydrocinnamyl acetate, linalyl acetate, octyl acetate, phenylethyl acetate, terpinyl acetate, triacetin, potassium acetate, sodium acetate, and calcium acetate, and esters and ester derivatives of GRAS flavor acids (c).

47. The medicament of claim 5, wherein the terpene is selected from the group consisting of camphor, limonene, and β-caryophyllene.

48. The medicament of claim 5, wherein the acetal is selected from the group consisting of acetal, acetaldehyde dibutyl acetal, acetaldehyde dipropyl acetal, acetaldehyde phenethyl propyl acetal, cinnamic aldehyde ethylene glycol acetal, decanal dimethyl acetal, heptanal dimethyl acetal, heptanal glyceryl acetal, and benzaldehyde propylene glycol acetal.

49. The medicament of claim 5, wherein the aldehyde is selected from the group consisting of anisaldehyde, benzaldehyde, iso-butyl aldehyde, citral, citronellal, n-caprylic aldehyde, ethylvanillin, furfural, heliotropin, heptyl aldehyde, hexyl aldehyde, 2-hexenal, hydrocinnamic aldehyde, lauryl aldehyde, nonyl aldehyde, octyl aldehyde, phenylacetaldehyde, propionaldehyde, vanillin, cinnamic aldehyde, perillaldehyde, and cuminaldehyde;

50. The medicament of claim 5, wherein the GRAS essential oil is selected from the group consisting of essential oils, alcoholic extracts, glycolic extracts, and extracts obtained by CO₂ high-pressure processes from a plant selected from the group consisting of melissa, coriander, cardamon, eucalyptus, cinnamon, lemon, lemon grass, citronella, lime, orange, origanum, thyme, rosemary, orange, clove, fennel, camphor, mandarin, anise, cascarilla, estragon, pimento, lavender, mustard, onion, garlic, pepper, bitter orange, caraway, dill, peppermint, nutmeg, and olibanum.

51. The medicament of claim 4, wherein the microbicidal composition further comprises at least one GRAS flavor alcohol (a) or derivative thereof, at least one polyphenol compound (b), or at least one GRAS flavor acid (c) or derivative thereof.

52. The medicament of claim 5, wherein said microbicidal composition further comprises from 0.001 to 25% by weight of flavoring agents (d) to (i).

53. The medicament of claim 8, wherein said microbicidal composition contains from 0.1 to 20% by weight of benzyl alcohol and from 0.01 to 10% by weight of tannin.

54. The medicament of claim 11, wherein said flavor carrier agents (j) are selected from the group consisting of propylene glycol, benzyl alcohol, glycerol, alginates, lactates, silica, and alginic acid.

55. The medicament of claim 13, wherein the microbicidal composition comprises from 0.1 to 100% by weight of GRAS essential oils (i), from 0 to 20% by weight of anisole, and from 0 to 20% by weight of quercitin.

56. The medicament of claim 14, wherein the aromatic GRAS flavor alcohol is benzyl alcohol.

57. The medicament of claim 16, wherein said microbicidal composition further comprises at least one of benzyl alcohol and a polyphenol compound (b).

58. The medicament of claim 19, wherein said microbicidal composition comprises at least two aromatic GRAS flavor alcohols.

59. The medicament of claim 58, wherein said microbicidal composition comprises two aromatic GRAS flavor alcohols or their derivatives.

60. The medicament of claim 19, wherein said lipophilic GRAS flavor alcohols are selected from the group consisting of benzyl alcohol, 2-phenylethanol, 1-phenyl ethanol, cinnamyl alcohol, hydrocinnamyl alcohol, 1-phenyl-1-propanol and anisalcohol, and said aliphatic GRAS flavor alcohols are selected from the group consisting of benzyl alcohol, n-butyl alcohol, iso-butyl alcohol, hexyl alcohol, L-menthol, octyl alcohol, heptyl alcohol, n-amyl alcohol, iso-amyl alcohol, anisalcohol, citronellol, n-decyl alcohol, geraniol, b-g-hexenol, lauryl alcohol, linalool, nerolidol, nonadienol, nonyl alcohol, rhodinol, terpineol, borneol, clineol, anisole, cuminyl alcohol, 10-undecene-1-ol, 1-hexadecanol, and derivatives thereof.

61. The medicament of claim 60, wherein said lipophilic GRAS flavor alcohol is benzyl alcohol.

62. The medicament of claim 20, wherein said monovalent or polyvalent alcohol has from 2 to 10 carbon atoms, said hydrophilic organic acid (cₕ) has from 2 to 10 carbon atoms, said hydrophilic acetate (eₕ) is selected from the group consisting of allicin, triacetin, potassium acetate, sodium acetate, and calcium acetate, and said hydrophilic aldehyde (hₕ) is selected from the group consisting of furfurol, propionaldehyde, and vanillin.

63. The medicament of claim 62, wherein the hydrophilic organic acid (cₕ) is selected from the group consisting of acetic acid, aconitic acid, formic acid, malic acid, lactic acid, phenylacetic acid, citric acid, mandelic acid, tartaric acid, fumaric acid, tannic acid, hydrocinnamic acid, and physiologically acceptable salts thereof.

64. The medicament of claim 20, wherein said monovalent or polyvalent alcohol has from 2 to 7 carbon atoms.

65. The medicament of claim 20, wherein the monovalent or polyvalent alcohol is selected from the group consisting of acetoin, ethyl alcohol, propyl alcohol, isopropyl alcohol, propylene glycol, and glycerol.

66. The medicament of claim 23, wherein the non-alcoholic hydrophilic GRAS flavoring agents are hydrophilic GRAS flavor acids (cₕ).

67. The medicament of claim 23, wherein the microbicidal composition comprises from 0.01 to 99% by weight of benzyl alcohol or a polyphenol compound (b) and from 0.01 to 50% by weight of a hydrophilic non-alcoholic GRAS flavor agent.

68. The medicament of claim 24, wherein the microbicidal composition comprises from 0.1 to 99% by weight of component (III), from 0 to 25% by weight of component (IV-1), and from 0 to 70% by weight of component (IV-2).

69. The medicament of claim 27, wherein component (III) of said microbicidal composition further comprises at least one lipophilic GRAS flavor alcohol (aₗ) or derivative thereof.

70. The medicament of claim 69, wherein the microbicidal composition comprises from 0.1 to 99% by weight of benzyl alcohol, from 0 to 99.8% by weight of component (aₗ), from 0 to 25% by weight of component (IV-1), and from 0 to 70% by weight of component (IV-2).

71. The medicament of claim 28, wherein component (IV) of said microbicidal composition further comprises at least one GRAS acid or a derivative thereof.

72. The medicament of claim 29, wherein the at least one GRAS flavoring agent (d) to (i) is present from 0.001 to 25% by weight in the microbicidal composition.

73. The medicament of claim 72, wherein the GRAS flavoring agent is selected form the group consisting of phenols (d) and essential oils (i).

74. The medicament of claim 30, wherein the polyphenol compound (b) is tannin.

75. The medicament of claim 74, wherein the microbicidal composition comprises from 20 to 98% by weight of benzyl alcohol and from 0.01 to 10% by weight of tannin.

76. The medicament of claim 31, wherein the at least one GRAS flavoring agent having one or more double bonds (Δ) is selected from the group consisting of GRAS acids (c_{Δ}), GRAS alcohols (a_{Δ}), and derivatives thereof.

77. The medicament according to claim 76, further comprising a GRAS acid (c_{Δ}) selected from the group consisting of cinnamic acid, fumaric acid, and derivatives thereof, and a GRAS alcohol (a_{Δ}) selected from the group consisting of cinnamyl alcohol, citronellol, 3-hexenol, nonadienol, 10-undecen-1-ol, and derivatives thereof, and wherein the composition contains from 0.01 to 70% by weight of said GRAS flavoring agents having double bonds.

78. The medicament of claim 1, wherein the medicament is in the form of an inhalation agent.

79. A method for preparing an agent selected from the group consisting of a decontaminative agent, a virucide, a toxin reduction agent, and a regenerative agent, the method comprising:
(a) providing the medicament of claim 1; and
(b) combining the medicament of claim 1 with a pharmaceutically acceptable carrier to prepare a decontaminative agent, a virucide, a toxin reduction agent, or a regenerative agent.

80. The method of claim 79, wherein the agent is selected from the group consisting of an antibiotic; a cytostatic agent; and an agent for the treatment of overweight, rheumatism, dermatoses, gastritis, gastro-intestinal diseases, bronchial diseases, depressions, arthritis, mucosal diseases, impotence, defective concentration, psychic disorders, migraine, sleep disturbance, gastro-intestinal symptoms, allergies and skin diseases, joint diseases, genital and hormonal disorders, infections, cancers, and immune insufficiency.

81. The method of claim 79, wherein the agent is in the form of an inhalation agent for the treatment of respiratory diseases in humans and animals.

82. The method of claim 42, wherein the subject is an animal.

83. The method of claim 82, wherein the animal is a human.
